(19) United States
(12) Patent Application Publication
(10) Pub. No.: US 2026/0242491 A1
(43) Pub. Date: Aug. 20, 2026

(54) IL-22R ANTIBODY FOR USE IN TREATING ATOPIC DERMATITIS

(71) Applicant: LEO PHARMA A/S, Ballerup (DK)

(72) Inventors: Klaus NIELSEN, Ballerup (DK); Stine Timmermann OTTESEN, Ballerup (DK)

(21) Appl. No.: 19/162,208

(22) PCT Filed: Mar. 5, 2024
(86) PCT No.: PCT/EP2024/055658

§ 371 Date: Sep. 4, 2025

(30) Foreign Application Priority Data
Mar. 8, 2023 (EP) 23160677.3
Apr. 19, 2023 (EP) 23168786.4

(51) Int. Cl. C07K 16/28; A61K 9/00; A61K 9/16; A61K 39/00; A61K 39/395; A61K 45/06; A61P 17/00

(52) U.S. Cl. CPC C07K 16/2866; A61K 9/0019; A61K 9/1617; A61K 9/1623; A61K 39/3955; A61K 45/06; A61P 17/00; A61K 2039/505; A61K 2039/54

(57) ABSTRACT

The present disclosure relates to the use of an IL-22R antibody for use in treating atopic dermatitis.

### FIELD OF THE INVENTION

The present invention relates to the use of an IL-22R antibody for use in treating atopic dermatitis.

### BACKGROUND OF THE INVENTION

IL-22R (also known as IL-22R1 and IL-22RA) is a type II cytokine receptor selectively expressed on skin and epithelial cells. This receptor mediates signaling via three cytokines: interleukin 22 (IL-22), interleukin 20 (IL-20) and interleukin 24 (IL-24). Cytokine signaling via the IL-22R requires the formation of heterodimeric complexes at the cell surface. IL-22 binds to and signals via a complex consisting of IL-22R and IL-10R3 (also known as IL-10R2), whereas IL-20 and IL-24 bind to and signal via a heterodimeric complex consisting of IL-22R and IL-20R3 (also known as IL-20R2). Interleukin-22 is a cytokine expressed by immune cells, particularly activated dendritic cells and T cells. Once produced by the immune system, IL-22 exerts its biological effects by binding to and activating IL-22R on epithelial cells. Activation of the IL-22R-IL-10R3 complex downstream of IL-22 binding leads to pro-inflammatory responses, the induction of anti-microbial proteins that are critical for host defense against bacterial pathogens, and protective effects in some organs such as the lungs and liver. IL-22 has also been implicated in disease pathology, particularly in the development of inflammatory disorders such as psoriasis, psoriatic arthritis and atopic dermatitis (Ma et al. J Clin. Invest. 1 18:597-607 (2008); Van Belle et al. J Immunol. January 1; 188 (1): 462-9 (2012); Sabat et al. Nat. Rev. Drug Discov. 13 (1): 21-38 (2014)).

The crystal structure of IL-22 in complex with the extracellular domain of IL-22R has been solved and has provided important insights into how this ligand associates with its receptor (Jones et al. Structure 16 (9): 1333-1344 (2008)). The extracellular region of IL-22R includes two fibronectin type III (FBNIII) domains (D1 and D2) oriented at approximately right angles to one another. Five loops located at the interface of these domains are primarily responsible for engaging IL-22 residues in the ligand-receptor complex. The IL-22 residues that contribute to receptor binding are clustered at two sites in the ligand, site 1a and site 1b. Insights into the critical residues contributed by both the receptor and the ligand have revealed ways in which this interaction could be disrupted to abrogate IL-22 signaling as a therapeutic strategy. Interleukin-20 and interleukin-24 are expressed by monocytes and keratinocytes and like IL-22, these cytokines have been found to play a role in skin homeostasis and pathology. It follows, that strategies to inhibit or reduce signaling downstream of IL-22R by blocking the binding of ligands that activate this receptor could have therapeutic utility, particularly in the treatment of skin conditions such as psoriasis and atopic dermatitis.

Antibodies that bind to IL-22R and block the interaction between IL-22 and IL-22R have been developed. For example, WO2011/061119 describes a humanized IL-22R antibody produced from a mouse anti-human monoclonal antibody describe in WO2006/047249.

Previous data have shown that targeting the IL-22 cytokine provides benefit in the severe subgroup of AD patients, Guttman-Yassky E, et al. J Am Acad Dermatol. 2018 May; 78 (5): 872-881.

### SUMMARY OF THE INVENTION

The inventors have found that administration on the IL-22R binding protein according to the invention to a subject suffering from moderate-to-severe atopic dermatitis provide early on-set of effect on atopic dermatitis over the first weeks of treatment, such as the first 6 weeks of treatment.

Thus, in one aspect the present invention relates to an IL-22R binding protein for use in a method of treating atopic dermatitis (AD) in a subject, wherein the method comprises the steps of: (a) administering subcutaneously one or more loading dose(s) of the IL-22R binding protein to the subject; and (b) administering subcutaneously one or more secondary dose(s) of the IL-22R binding protein to the subject, wherein the II22R binding protein is an antibody with a heavy chain comprising a HCDR1 having the amino acid sequence of SEQ ID no. 1 (SYDMN), a HCDR2 having the amino acid sequence of SEQ ID no.2 (SIYNDASNTAYSDSVKG), a HCDR3 having the amino acid sequence of SEQ ID no.3 (VGFSGTYYSES), and a light chain comprising a LCDR1 having the amino acid sequence of SEQ ID no 4 (QGGYYAH), a LCDR2 having the amino acid sequence of SEQ ID No 5 (GQNNRPS) and a LCDR3 having the amino acid sequence of SEQ ID no 6 (QSGSSSSNAV).

In another aspect, the invention relates to a method of treating atopic dermatitis (AD) in a subject in need thereof, wherein the method comprises the steps of: (a) administering subcutaneously one or more loading dose(s) of an IL-22R binding protein to the subject; and (b) administering subcutaneously one or more secondary dose(s) of the IL-22R binding protein to the subject, wherein the II22R binding protein is an antibody with a heavy chain comprising a HCDR1 having the amino acid sequence of SEQ ID no. 1 (SYDMN), a HCDR2 having the amino acid sequence of SEQ ID no.2 (SIYNDASNTAYSDSVKG), a HCDR3 having the amino acid sequence of SEQ ID no.3 (VGFSGTYYSES), and a light chain comprising a LCDR1 having the amino acid sequence of SEQ ID no 4 (QGGYYAH), a LCDR2 having the amino acid sequence of SEQ ID No 5 (GQNNRPS) and a LCDR3 having the amino acid sequence of SEQ ID no 6 (QSGSSSSNAV).

In a Third aspect the invention relates to the use of an IL-22R binding protein in the manufacture of a medicament for treating atopic dermatitis (AD) in a subject, wherein the method for treating AD comprises the steps of: (a) administering subcutaneously one or more loading dose(s) of the IL-22R binding protein to the subject; and (b) administering subcutaneously one or more secondary dose(s) of the IL-22R binding protein to the subject, wherein the II22R binding protein is an antibody with a heavy chain comprising a HCDR1 having the amino acid sequence of SEQ ID no. 1 (SYDMN), a HCDR2 having the amino acid sequence of SEQ ID no.2 (SIYNDASNTAYSDSVKG), a HCDR3 having the amino acid sequence of SEQ ID no.3 (VGFSGTYYSES), and a light chain comprising a LCDR1 having the amino acid sequence of SEQ ID no 4 (QGGYYAH), a LCDR2 having the amino acid sequence of SEQ ID No 5 (GQNNRPS) and a LCDR3 having the amino acid sequence of SEQ ID no 6 (QSGSSSSNAV).

### DETAILED DESCRIPTION OF THE INVENTION

Unless defined otherwise, all technical and scientific terms used herein have the same meaning as commonly understood to one of ordinary skill in the art to which this invention belongs.

The articles “a” and “an” refer to one or to more than one (i.e., to at least one) of the grammatical object of the article. By way of example, “an element” means one element or more than one element.

The terms “comprise” and “comprising” are used in the inclusive, open sense, meaning that additional elements may be included.

In general, methods “comprising” a number of steps do not require the steps to be performed in a particular order. Where a method comprises a number of sequentially numbered or alphabetical steps (e.g. (1), (2), (3); (i), (ii), (iii); or (a), (b), (c) etc.), this implies that the steps must be performed in the prescribed order unless stated otherwise.

The term “including” is used herein to mean “including but not limited to”.

The term “about” in relation to a numerical value x is optional and means, for example, x±10%.

Generally, the terms “treat”, “treating”, “treatment”, or the like, mean to alleviate (reduce, minimise, or eliminate) symptoms, or to reduce, minimise or eliminate the causation of symptoms either on a temporary or permanent basis.

All publications mentioned herein are incorporated by reference in their entirety.

As used herein, the term “subject”, “individual” or “patient” is used interchangeably, refers to any animal, including mammals, preferably mice, rats, other rodents, rabbits, dogs, cats, swine, cattle, sheep, horses, or primates, and most preferably humans. In some embodiments as above, the patient is a human. In some embodiments as above, the patient is a human aged 12 years or older.

In some embodiments as above “patients” means one patient. In some embodiments as above “patients” means a patient population. In some embodiments as above “patients” means one or more patients.

As used herein “contains” is equivalent to “comprises”.

### Abbreviations

ADSD=Atopic Dermatitis Symptom Diary;
DLQI=Dermatology Life Quality Index;
eDiary=electronic diary;
EQ-5D-5L=EuroQol 5-Dimension Health Questionnaire 5 Level;
HADS=Hospital Anxiety and Depression Scale;
PGI-C=Patient Global Impression of Change;
PGI-S=Patient Global Impression of Severity;
POEM=Patient-Oriented
Eczema Measure;
PRO=patient-reported outcome;
WPAI-SHP=Work Productivity and
Activity Impairment: Specific Health Problem.
VL=light chain variable domain
VH=heavy chain variable domain
HC=heavy chain
HL=light chain

### FIGURES

FIG. 1: Change in EASI score over 16 weeks of treatment according to the trial outlined in Example 1.

FIG. 2: Responders at week 16

FIG. 3: Overall summary of AEs at week 16

### SEQUENCES

The IL-22 receptor antibody used according to the invention is described in WO2018011420 as antibody 230C9. The antibody used according to the invention is defined therein by the HC of seq. id. No. 67 and LC of seq. id. No 68, the VH by sequence no. 63 and VL by sequence no 64, HCDR1 sequence no. 34 (SYDMN), HCDR2 sequence no. 36 (SIYNDASNTAYSDSVKG) and HCDR3 sequence no. 6 (VGFSGTYYSES). LCDR1 sequence no. 16 (QGGYYAH), LCDR2 sequence no. 47 (GQNNRPS) and LCDR3 sequence no. 54 (QSGSSSSNAV). Sequence numbers refers to the numbers of the application above. Below the sequences no. 67, 68, 64 and 63 are outlined.

| | |
|---|---|
| 67 | 1 QVQLVESGGG LVQPGGSLRL SCAASGFTFS SYDMNWVRQA PGKGLEWVSS |
| | 51 IYNDASNTAY SDSVKGRFTI SRDNSKNTLY LQMNSLRAED TAVYYCAKVG |
| | 101 FSGTYYSESW GQGTLVTVSS ASTKGPSVFP LAPSSKSTSG GTAALGCLVK |
| | 151 DYFPEPVTVS WNSGALTSGV HTFPAVLQSS GLYSLSSVVT VPSSSLGTQT |
| | 201 YICNVNHKPS NTKVDKKVEP KSCDKTHTCP PCPAPELLGG PSVFLFPPKP |
| | 251 KDTLMISRTP EVTCVVVDVS HEDPEVKFNW YVDGVEVHNA KTKPREEQYQ |
| | 301 STYRVVSVLT VLHQDWLNGK EYKCKVSNKA LPAPIEKTIS KAKGQPREPQ |
| | 351 VYTLPPSRDE LTKNQVSLTC LVKGFYPSDI AVEWESNGQP ENNYKTTPPV |
| | 401 LDSDGSFFLY SKLTVDKSRW QQGNVFSCSV MHEALHNHYT QKSLSLSPGK |
| 68 | 1 SYELTQPSSV SVALGQTARI TCQGGYYAHW YQQKPGQAPV LVIYGQNNRP |
| | 51 SGIPERFSGS GAGNTATLTI SRAQAEDEAD YYCQSGSSSS NAVFGGGTKL |
| | 101 TVLGQPKAAP SVTLFPPSSE ELQANKATLV CLISDFYPGA VTVAWKADSS |
| | 151 PVKAGVETTT PSKQSNNKYA ASSYLSLTPE QWKSHRSYSC QVTHEGSTVE |
| | 201 KTVAPTECS |
| 64 | 1 SYELTQPSSV SVALGQTARI TCQGGYYAHW YQQKPGQAPV LVIYGQNNRP |
| | 51 SGIPERFSGS GAGNTATLTI SRAQAEDEAD YYCQSGSSSS NAVFGGGTKL |
| | 101 TVL |
| 63 | 1 QVQLVESGGG LVQPGGSLRL SCAASGFTFS SYDMNWVRQA PGKGLEWVSS |
| | 51 IYNDASNTAY SDSVKGRFTI SRDNSKNTLY LQMNSLRAED TAVYYCAKVG |
| | 101 FSGTYYSESW GQGTLVTVSS |

The antibody is also described as anti-IL-22R, IL-22 receptor antibody, the antibody binding to IL-22 receptor.

A functional variant (equivalent) of IL22R antibody as above, which has essentially the same epitope-binding specificity as anti-IL-22R and exhibits substantially similar bioactivity, is also included in the scope of the present invention and also disclosed in WO2018011420. In some embodiments, a functional variant contains the same regions/residues responsible for antigen-binding, such as the same specificity-determining residues in the CDRs or the whole CDRs. In other embodiments, a functional variant comprises a VH chain that includes a HCDR1, HCDR2, and HCDR3 at least 75% (e.g., 80%, 85%, 15 90%, 95%, or 98%) identical to the corresponding HCDRs of the antibody above, and a VL chain that includes a LCDR1, LCDR2, and LCDR3 at least 75% (e.g., 80%, 85%, 90%, 95%, or 98%) identical to the corresponding LCDRs as mentioned above. For example, a functional variant may comprise a VH chain that includes up to 5 (e.g., 1, 2, 3, 4, or 5) amino acid residue variations in the HCDR regions (HCDR1, CDR2, and/or CDR3 in total) as compared to the HCDRs disclosed above, and/or a VL chain that includes up to 5 (e.g., 1, 2, 3, 4, or 5) amino acid residue variations in the LCDR regions (LCDR1, CDR2, and/or CDR3 in total) as compared to the LCDRs as mentioned above. Alternatively, a functional variant comprises a VH chain at least 75% (e.g., 80%, 85%, 90%, 95%, or 98%) identical to the VH chain as defined above and a VL chain at least 75% 25 (e.g., 80%, 85%, 90%, 95%, or 98%) identical to the VL chain as defined above. The amino acid sequence variations may occur only in one or more of the VH and/or VL framework regions. It is anticipated that antibodies having essentially same characteristics as the above can be useful in the present invention. Some sequence variation while still maintaining the binding characteristics are variations which are covered by the present invention.

Alternatively, or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for 10 altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include 15 substitutions made amongst amino acids within the following groups: (a) M, I, L, V; (b) F, Y, W; (c) K, R, H; (d) A, G; (e) S, T; (f) Q, N; and (g) E, D.

### Atopic Dermatitis Disease Severity Outcome Measures

### EASI

The EASI is a validated measure used in clinical practice and clinical trials to assess the severity of AD (Hanifin JM, Thurston M, Omoto M, Cherill R, Tofte SJ, Graeber M. The eczema area and severity index (EASI): assessment of reliability in atopic dermatitis. EASI Evaluator Group. Exp Dermatol. 2001; 10 (1): 11-18).

The assessment will be based on the condition of the disease at the time of evaluation and not in relation to the condition at a previous visit. The scoring is based on the Harmonising Outcome Measures for Eczema (http://www.homeforeczema.org/) EASI guidance version 3. The EASI is a composite index with scores ranging from 0 to 72, with higher values indicating more severe or more extensive condition. The index will be calculated as shown in Table 1. Briefly, the investigator will assess the severity of 4 AD disease characteristics (erythema, edema/papulation, excoriation, and lichenification) on the 4 body regions (head/neck, trunk, upper extremities, lower extremities); severity will be assessed according to the scale shown in Table 2. For each body region, a severity sum score will be calculated which will be multiplied by an area score (Table 2) and by a weighting factor. The EASI score equals the sum of the scores obtained for each body region (Table 1).

TABLE 1
Calculation of the EASI score

| | | | | | | | |
|---|---|---|---|---|---|---|---|
| | | Edema/ | | | Area | Weighting | |
| Body region | Erythema | papulation | Excoriation | Lichenification | score | factor | Score |

| | | | | | | |
|---|---|---|---|---|---|---|
| Head/neck | (SS+ | SS+ | SS+ | SS) | ×AS | ×0.1 |
| Trunk | (SS+ | SS+ | SS+ | SS) | ×AS | ×0.3 |
| Upper extremities | (SS+ | SS+ | SS+ | SS) | ×AS | ×0.2 |
| Lower extremities | (SS+ | SS+ | SS+ | SS) | ×AS | ×0.4 |

| | |
|---|---|
| The EASI score is the sum of the 4 body region scores | (Range |
| | 0-72) |

Abbreviations: AS = area score; EASI = Eczema Area and Severity Index; SS = severity score. Modified from (HOME. Harmonising Outcome Measures for Eczema. Eczema Area and Severity Index (EASI) case report form. Aged 8 and over. 2017).

TABLE 2
EASI severity score scale and area score scale

| | |
|---|---|
| Severity score scale | Area score scale |

| | | | |
|---|---|---|---|
| 0 | None/absent | 0 | 0% affected area |
| 1 | Mild | 1 | 1% to 9% affected area |
| 2 | Moderate | 2 | 10% to 29% affected area |
| 3 | Severe | 3 | 30% to 49% affected area |
| | | 4 | 50% to 69% affected area |
| | | 5 | 70% to 89% affected area |
| | | 6 | 90% to 100% affected area |

Note:
half-points (1.5 and 2.5) are allowed. 0.5 is not permitted.
Abbreviations: EASI = Eczema Area and Severity Index.

### SCORAD

The SCORAD is a validated tool to evaluate the extent and severity of AD lesions, along with subjective symptoms (Severity scoring of atopic dermatitis: the SCORAD index. Consensus Report of the European Task Force on Atopic Dermatitis. Dermatology. 1993; 186 (1): 23-31.) The maximum total score is 103, with higher values indicating more severe disease.

The assessment consists of 3 components: A=extent, B=intensity, and C=subjective symptoms.

### Extent (A)

The extent of AD is assessed as a percentage of each defined body area and reported as the sum of all areas (maximum score=100%).

### Intensity (B)

The intensity of 6 specific symptoms of AD (erythema, edema/papulation, oozing/crusting, excoriation, lichenification, and dryness) is assessed by the investigator on an average representative area using the following scale:

$$0 = None / absent$$

$$1 = Mild$$

$$2 = Moderate$$

$$3 = Severe$$

Note: dryness is evaluated on uninvolved areas.

The sum of intensity score of the 6 symptoms will be reported (maximum score=18).

### Subjective Symptoms (C)

A subjective assessment of the average pruritus and sleep loss over the last 3 days/nights is recorded for each symptom by the subject on a VAS, where 0 is no itching (or no trouble sleeping) and 10 is unbearable itching (or lot of trouble sleeping), with a maximum possible score of 20.

The SCORAD is calculated as: A/5+7B/2+C.

vIGA-AD

The vIGA-AD is an instrument used in clinical trials to assess the subject's global disease severity and is based on a 5-point scale ranging from 0 (clear) to 4 (severe) (Table 3) (Eli Lilly and Company. Validated Investigator Global Assessment scale for Atopic Dermatitis. vIGA-AD™. 2017.). The vIGA-AD score will be assessed according to the schedule of trial procedures. The assessment is based on the condition of the disease at the time of evaluation and not in relation to the condition at a previous visit.

TABLE 3
vIGA-AD

| Score | Morphological description |
|---|---|
| 0 - Clear | No inflammatory signs of AD (no erythema, no induration/ |
| | papulation, no lichenification, no oozing/crusting). Post- |
| | inflammatory hyperpigmentation and/or hypopigmentation |
| | may be present. |
| 1 - Almost | Barely perceptible erythema, and barely perceptible |
| clear | induration/papulation, and/or minimal lichenification. No |
| | oozing or crusting. |
| 2 - Mild | Slight but definite erythema (pink), slight but definite |
| | induration/papulation, and/or slight but definite |
| | lichenification. No oozing or crusting. |
| 3 - Moderate | Clearly perceptible erythema (dull red), clearly perceptible |
| | induration/papulation, and/or clearly perceptible |
| | lichenification. Oozing and crusting may be present. |
| 4 - Severe | Marked erythema (deep or bright red), marked induration/ |
| | papulation, and/or marked lichenification. Disease is |
| | widespread in extent. Oozing or crusting may be present. |

### BSA Involvement

The total BSA affected by AD will be assessed by the investigator for each section of the body as component A of SCORAD and will be reported as a percentage of all major body sections combined. The following body regions will be assessed (brackets show the highest possible score for each region): head and neck (9%), anterior trunk (18%), back (18%), upper limbs (18%), lower limbs (36%), and genitals (1%).

The investigator will assess the total AD involvement for the whole body, i.e., head/neck, upper limbs, trunk, genitalia, and lower limbs, as a percentage of the total BSA. As a guidance for this estimate, the surface of a full, flat palm (including the 5 fingers) of an adult subject corresponds to approximately 1% of the total BSA.

### PROs

The subject will be asked to complete PROs according to the schedule of trial. PROs will either be completed at home (eDiary) or at the trial site (electronic device) (Table 4).

TABLE 4
Overview of PROs to be completed

| | PROs to be completed at the |
|---|---|
| PROs to be completed in the eDiary | trial site |
| To be completed in the morning⁰ | DLQI |
| Nocturnal Scratching | POEM |
| Difficulty Falling Asleep | EQ-5D-5L |
| Frequency of Waking up During | WPAI-SHP |
| the Night | |
| To be completed in the evening⁰ | HADS |
| ADSD (all components) | PGI-S |
| | Itch, Skin Pain, ADSD, Nocturnal |
| | Scratching, |
| | Difficulty Falling Asleep, |
| | Frequency of Waking up During |
| | the Night |
| | PGI-C |
| | Itch, Skin Pain, ADSD, Nocturnal |
| | Scratching, |
| | Difficulty Falling Asleep, |
| | Frequency of Waking up During |
| | the Night |

(a)The morning assessments will only be completed until Week 32, the evening assessments will be completed until Week 48.

Nocturnal Scratching (eDiary)

Nocturnal Scratching is a single item questionnaire designed developed by LEO Pharma to assess how much the subject scratched due to itchy skin during the past night. It will be assessed on a 6-point categorical scale (‘none of the night’, ‘a little of the night’, ‘some of the night’, ‘a lot of the night’, ‘all of the night’, ‘I don't know’).

Difficulty Falling Asleep (eDiary)

Difficulty Falling Asleep is a single item questionnaire designed developed by LEO Pharma to assess the subject's difficulty to fall asleep because of itchy skin the past night. It will be assessed on a 5-point categorical scale (‘not difficult’, ‘a little difficult’, ‘moderately difficult’, ‘extremely difficult’, ‘I don't know’).

Frequency of Waking Up During the Night (eDiary)

Frequency of Waking up During the Night is a single item questionnaire designed developed by LEO Pharma to assess how many times the subject woke up because of itchy skin the past night. It will be assessed on a 5-point categorical scale (‘not at all’, ‘woke up 1 time’, ‘woke up 2 times’, ‘woke up 3 times or more’, ‘I don't know’).

ADSD (eDiary)

ADSD is considered a subject's assessment of efficacy. ADSD is a 9-item sign and symptom diary developed by LEO Pharma. It assesses the severity of itch, painful skin, red skin, dry skin, rough skin, flaky skin, cracked skin, hard skin, and swollen skin. The subject assesses the severity of each symptom ‘at its worst’ over the past 24 hours using an 11-point numeric rating scale, with anchors at 0=‘no [symptom]’ and 10=‘worst possible [symptom]’.

### DLQI (at Trial Site)

DLQI consists of 10 items addressing the subject's perception of the impact of their skin disease on different aspects of their quality of life over the past week such as dermatology related symptoms and feelings, daily activities, leisure, work or school, personal relationships, and the treatment. Each item is scored on a 4 point Likert scale (0=‘not at all/not relevant’, 1=‘a little’, 2=‘a lot’, 3=‘very much’). The total score (0 to 30) is the sum of the 10 items with higher scores indicating a poorer quality of life (Finlay AY, Khan GK. Dermatology Life Quality Index (DLQI)—a simple practical measure for routine clinical use. Clin Exp Dermatol. 1994; 19 (3): 210-216.).

### POEM (at Trial Site)

POEM consists of 7 items, each addressing a specific symptom (itching, sleep, bleeding, weeping, cracking, flaking, and dryness). The subject will be asked to score how often they experienced each symptom over the past week on a 5 point categorical scale (0=‘no days’, 1=‘1 to 2 days’, 2=‘3 to 4 days’, 3=‘5 to 6 days’, 4=‘every day’). The total score (0 to 28) is the sum of the 7 items and reflects disease related morbidity with higher scores indicating more severe symptoms (Charman CR, Venn AJ, Williams HC. The patient-oriented eczema measure: development and initial validation of a new tool for measuring atopic eczema severity from the patients' perspective. Arch Dermatol. 2004; 140 (12): 1513-1519).

### EQ-5D-5L (at Trial Site)

EQ-5D-5L is a self-administered questionnaire used to assess health status ‘today’ and is divided into 2 sections. The first section includes 5 dimensions (mobility, self-care, usual activity, pain/discomfort, and anxiety/depression). Each dimension is assessed using a 5-point categorical scale (‘no problems’, ‘slight problems’, ‘moderate problems’, ‘severe problems’, and ‘extreme problems’). The second section consists of a vertical VAS anchored at 0 (‘the worst health you can imagine’) and 100 (‘the best health you can imagine’) (EuroQol—a new facility for the measurement of health-related quality of life. Health Policy. 1990; 16 (3): 199-208.).

### WPAI-SHP (at Trial Site)

WPAI-SHP consists of 6 items and scores can be calculated for 4 domains, each reflecting the percentage impairment due to AD during the past week, with higher scores indicating a greater impairment and lesser productivity (Reilly MC, Zbrozek AS, Dukes EM. The validity and reproducibility of a work productivity and activity impairment instrument. Pharmacoeconomics. 1993; 4 (5): 353-365.):
Absenteeism: percentage work time missed due to AD for those who were employed the past week.
Presenteeism: percentage impairment while working due to AD for those who were employed and actually worked the past week.
Work productivity loss: percentage overall work impairment due to AD for those who were employed the past week.
Activity impairment: percentage activity impairment due to AD for all respondents.

### HADS (at Trial Site)

HADS is a 14-item questionnaire with scores ranging from 0 to 21 for anxiety and 0 to 21 for depression. Each of the 14 items will be evaluated on a Likert scale. Scores are considered normal from 0 to 7 and abnormal from 11 to 21 and are categorized as: normal (0 to 7), mild (8 to 10), moderate (11 to 14), and severe (15 to 21) (Zigmond AS, Snaith RP. The hospital anxiety and depression scale. Acta Psychiatr Scand. 1983; 67 (6): 361-370.).

### PGI-S (at Trial Site)

PGI-S is a questionnaire designed to assess the subject's overall perception of severity. In this trial the severity of itch, skin pain, ADSD, nocturnal scratching, difficulty falling asleep, and frequency of waking up during the night over the past week will be evaluated. The subject will be asked to choose 1 option from a 4-point categorical scale as described in Table 5 (FDA. Food and Drug Administration. Patient-Focused Drug Development Guidance Public Workshop. Methods to Identify What is Important to Patients & Select, Develop or Modify Fit-for-Purpose Clinical Outcomes Assessments. 15-16 Oct. 2018.).

TABLE 5

| PGI-S | Scale |
|---|---|
| Itch | ‘None’, ‘mild’, ‘moderate’, |
| Skin Pain | ‘severe’ |
| ADSD | |
| Nocturnal Scratching | |
| Difficulty Falling Asleep | |
| Frequency of Waking up During the Night | ‘Never’, ‘sometimes’, ‘often’, |
| PGI-S | ‘very often’ |

Abbreviations: ADSD = atopic dermatitis symptom diary; PGI-S = Patient's Global Impression of Severity.

### PGI-C (at Trial Site)

PGI-C is a questionnaire designed to assess the subject's overall impression of change. In this trial the change in itch, skin pain, ADSD, nocturnal scratching, difficulty falling asleep, and frequency of waking up during the night since the subject started treatment will be evaluated. The subject will be asked to choose 1 option from a 5 point categorical scale (‘much better’, ‘a little better’, ‘no change’, ‘a little worse’, ‘much worse’) (FDA. Food and Drug Administration. Patient-Focused Drug Development Guidance PublicWorkshop. Methods to Identify What is Important to Patients & Select, Develop or Modify Fit-for-Purpose Clinical Outcomes Assessments. 15-16 Oct. 2018.)

### FURTHER EMBODIMENTS

In one embodiment the invention relates to the IL-22R binding protein for use, the method or the use as above, wherein the II22R binding is an antibody comprises a VH having the amino acid sequence of SEQ ID NO 7 (QVQLVESGGG LVQPGGSLRL SCAASGFTFS SYDMNWVRQA PGKGLEWVSS IYNDASNTAY SDSVKGRFTI SRDNSKNTLY LQMNSLRAED TAVYYCAKVG FSGTYYSESW GQGTLVTVSS) and a VL having the amino acid sequence of SEQ ID NO. 8 (SYELTQPSSV SVALGQTARI TCQGGYYAHW YQQKPGQAPV LVIYGQNNRP SGIPERFSGS GAGNTATLTI SRAQAEDEAD YYCQSGSSSS NAVFGGGTKLTVL).

In one embodiment the invention relates to the IL-22R binding protein for use, the method or the use as above, wherein the II22R binding is an antibody comprises a HC having the amino acid sequence of SEQ ID NO 9 (QVQLVESGGG LVQPGGSLRL SCAASGFTFS SYDMNWVRQA PGKGLEWVSS IYNDASNTAY SDSVKGRFTI SRDNSKNTLY LQMNSLRAED TAVYYCAKVG FSGTYYSESW GQGTLVTVSS ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKVEP KSCDKTHTCP PCPAPELLGG PSVFLFPPKP KDTLMISRTP EVTCVVVDVS HEDPEVKFNW YVDGVEVHNA KTKPREEQYQ STYRVVSVLT VLHQDWLNGK EYKCKVSNKA LPAPIEKTIS KAKGQPREPQ VYTLPPSRDE LTKNQVSLTC LVKGFYPSDI AVEWESNGQP ENNYKTTPPV LDSDGSFFLY SKLTVDKSRW QQGNVFSCSV MHEALHNHYT QKSLSLSPGK) and a LC having the amino acid sequence of SEQ ID NO. 10 (SYELTQPSSV SVALGQTARI TCQGGYYAHW YQQKPGQAPV LVIYGQNNRP SGIPERFSGS GAGNTATLTI SRAQAEDEAD YYCQSGSSSS NAVFGGGTKL TVLGQPKAAP SVTLFPPSSE ELQANKATLV CLISDFYPGA VTVAWKADSS PVKAGVETTT PSKQSNNKYA ASSYLSLTPE QWKSHRSYSC QVTHEGSTVE KTVAPTECS).

In one embodiment the invention relates to the IL-22R binding protein for use, the method or the use as above, wherein the method comprises the steps of:
(a) administering one or more loading dose(s) of about 600 mg of the IL-22R binding protein to the subject; and (b) administering one or more secondary dose(s) of about 600 mg of the IL-22R binding protein to the subject, wherein each secondary dose is administered to the subject about 2 weeks after the immediately preceding dose;
(b) administering one or more loading dose(s) of about 450 mg of the IL-22R binding protein to the subject; and (b) administering one or more secondary dose(s) of about 450 mg of the IL-22R binding protein to the subject, wherein each secondary dose is administered to the subject about 2 weeks after the immediately preceding dose;
(c) administering one or more loading dose(s) of about 600 mg of the IL-22R binding protein to the subject; and (b) administering one or more secondary dose(s) of about 300 mg of the IL-22R binding protein to the subject, wherein each secondary dose is administered to the subject about 2 weeks after the immediately preceding dose; or
(d) administering one or more loading dose(s) of about 300 mg of the IL-22R binding protein to the subject; and (b) administering one or more secondary dose(s) of about 150 mg of the IL-22R binding protein to the subject, wherein each secondary dose is administered to the subject about 2 weeks after the immediately preceding dose.

In one embodiment the invention relates to the IL-22R binding protein for use, the method or the use as above, wherein the method comprises the steps of:
(a) administering five loading dose(s) of about 600 mg of the IL-22R binding protein to the subject; and (b) administering one or more secondary dose(s) of about 600 mg of the IL-22R binding protein to the subject, wherein each loading dose after the first loading dose is administered to the subject about 1 week after the immediately preceding loading dose and each secondary dose is administered to the subject about 2 weeks after the immediately preceding dose;
(b) administering three loading dose(s) of about 450 mg of the IL-22R binding protein to the subject; and (b) administering one or more secondary dose(s) of about 450 mg of the IL-22R binding protein to the subject, wherein each loading dose is administered to the subject about 1 week after the immediately preceding loading dose and wherein each secondary dose is administered to the subject about 2 weeks after the immediately preceding dose;
(c) administering two loading doses of about 600 mg of the IL-22R binding protein to the subject; and (b) administering one or more secondary dose(s) of about 300 mg of the IL-22R binding protein to the subject, wherein the second loading dose is administered to the subject about 2 weeks after the first loading dose and wherein each secondary dose is administered to the subject about 2 weeks after the immediately preceding dose; or
(d) administering two loading doses of about 300 mg of the IL-22R binding protein to the subject; and (b) administering one or more secondary dose(s) of about 150 mg of the IL-22R binding protein to the subject, wherein the second loading dose is administered to the subject about 2 weeks after the first loading dose and wherein each secondary dose is administered to the subject about 2 weeks after the immediately preceding dose.

In one embodiment the invention relates to the IL-22R binding protein for use, the method or the use as above, wherein the method comprises the steps of:
(a) administering five loading dose(s) of about 600 mg of the IL-22R binding protein to the subject; and (b) administering one or more secondary dose(s) of about 600 mg of the IL-22R binding protein to the subject, wherein each loading dose after the first loading dose is administered to the subject about 1 week after the immediately preceding dose and each secondary dose is administered to the subject about 2 weeks after the immediately preceding dose;
(b) administering three loading dose(s) of about 450 mg of the IL-22R binding protein to the subject; and (b) administering one or more secondary dose(s) of about 450 mg of the IL-22R binding protein to the subject, wherein the loading dose is administered to the subject about 1 week after the immediately preceding loading dose and wherein each secondary dose is administered to the subject about 2 weeks after the immediately preceding dose;
(c) administering two loading doses of about 600 mg of the IL-22R binding protein to the subject; and (b) administering one or more secondary dose(s) of about 300 mg of the IL-22R binding protein to the subject, wherein the second loading dose is administered to the subject about 1 week after the first loading dose and wherein each secondary dose is administered to the subject about 2 weeks after the immediately preceding dose; or
(d) administering two loading doses of about 300 mg of the IL-22R binding protein to the subject; and (b) administering one or more secondary dose(s) of about 150 mg of the IL-22R binding protein to the subject, wherein the second loading dose is administered to the subject about 1 week after the first loading dose and wherein each secondary dose is administered to the subject about 2 weeks after the immediately preceding dose.

In one embodiment the invention relates to the IL-22R binding protein for use, the method or the use as above, wherein the method comprises the steps of:
(a) administering five loading dose(s) of about 600 mg of the IL-22R binding protein to the subject; and (b) administering one or more secondary dose(s) of about 600 mg of the IL-22R binding protein to the subject, wherein each loading after the first loading dose is administered to the subject about 1 week after the immediately preceding dose and each secondary dose is administered to the subject about 2 weeks after the immediately preceding dose;
(b) administering three loading dose(s) of about 450 mg of the IL-22R binding protein to the subject; and (b) administering one or more secondary dose(s) of about 450 mg of the IL-22R binding protein to the subject, wherein the second loading dose is administered to the subject about 1 weeks after the first loading dose and wherein each secondary dose is administered to the subject about 2 weeks after the immediately preceding dose;
(c) administering two loading doses of about 600 mg of the IL-22R binding protein to the subject; and (b) administering one or more secondary dose(s) of about 300 mg of the IL-22R binding protein to the subject, wherein each secondary dose is administered to the subject about 2 weeks after the immediately preceding dose; or
(d) administering two loading doses of about 300 mg of the IL-22R binding protein to the subject; and (b) administering one or more secondary dose(s) of about 150 mg of the IL-22R binding protein to the subject, wherein the second loading dose is administered to the subject about 1 week after the first loading dose and wherein each secondary dose is administered to the subject about 4 weeks after the immediately preceding dose.

In one embodiment the invention relates to the IL-22R binding protein for use, the method or the use as above, wherein the method comprises the steps of:
(a) administering seven loading dose(s) of about 450 mg of the IL-22R binding protein to the subject; and (b) administering one or more secondary dose(s) of about 450 mg of the IL-22R binding protein to the subject, wherein each loading after the first loading dose is administered to the subject about 1 week after the immediately preceding dose and each secondary dose is administered to the subject about 2 weeks after the immediately preceding dose;
(b) administering two loading dose(s) of about 450 mg of the IL-22R binding protein to the subject; and (b) administering one or more secondary dose(s) of about 450 mg of the IL-22R binding protein to the subject, wherein the second loading dose is administered to the subject about 1 weeks after the first loading dose and wherein each secondary dose is administered to the subject about 2 weeks after the immediately preceding dose;
(c) administering one loading doses of about 450 mg of the IL-22R binding protein to the subject; and (b) administering one or more secondary dose(s) of 225300 mg of the IL-22R binding protein to the subject, wherein each secondary dose is administered to the subject about 2 weeks after the immediately preceding dose; or
(d) administering two loading doses of about 225 mg of the IL-22R binding protein to the subject; and (b) administering one or more secondary dose(s) of about 225 mg of the IL-22R binding protein to the subject, wherein the second loading dose is administered to the subject about 1 week after the first loading dose, the first secondary dose is administered week 4 and wherein the following secondary doses is administered to the subject about 4 weeks after the immediately preceding dose.

In one embodiment the invention relates to the IL-22R binding protein for use, the method or the use as above, wherein step (b) is continued for at least 8 weeks, at least 12 weeks, at least 3 months, at least 16 weeks, at least 20 weeks, at least 24 weeks, at least 6 months, at least 28 weeks, at least 32 weeks, at least 36 weeks, at least a year, or at least 52 weeks or more.

In one embodiment the invention relates to the IL-22R binding protein for use, the method or the use as above, wherein the subject's has moderate to severe atopic dermatitis as defined by the Hanifin and Rajka (1980) criteria for AD and a history for AD≥1 year.

In one embodiment the invention relates to the IL-22R binding protein for use, the method or the use as above, wherein the patient has

$$EASI score \geq 16 at baseline ,$$

$$vIGA - AD score \geq 3 at baseline ,$$

BSA of AD involvement≥10 at baseline,
and/or
ADSD worst itch score (weekly average)≥4 at baseline.

In one embodiment the invention relates to the IL-22R binding protein for use, the method or the use as above, wherein the subject has a recent history (within 12 months before screening) with documented inadequate response to treatment with TCS (±TCI as appropriate) or for whom these topical AD treatments are medically inadvisable e.g. due to important side effects or safety risks

In one embodiment the invention relates to the IL-22R binding protein for use, the method or the use as above, wherein administration of the IL-22R antibody eliminates or reduces disease severity and wherein the disease severity is assessed by an Atopic Dermatitis Disease Severity Outcome Measure.

In one embodiment the invention relates to the IL-22R binding protein for use, the method or the use as above, wherein Atopic Dermatitis Disease Severity Outcome Measure is Eczema Area and Severity Index (EASI) and EASI score is reduced from baseline.

In one embodiment the invention relates to the IL-22R binding protein for use, the method or the use as above, wherein Atopic Dermatitis Disease Severity Outcome Measure is Eczema Area and Severity Index (EASI) and the subjects EASI score is reduced from baseline at week 16.

In one embodiment the invention relates to the IL-22R binding protein for use, the method or the use as above, wherein Atopic Dermatitis Disease Severity Outcome Measure is the Validated Investigator Global Assessment Scale for Atopic Dermatitis (vIGA-AD) and wherein the subjects have achieved a vIGA-AD score of 0 (clear) or 1 (almost clear) at week 16 or earlier.

In one embodiment the invention relates to the IL-22R binding protein for use, the method or the use as above, wherein Atopic Dermatitis Disease Severity Outcome Measure is Severity Scoring of Atopic Dermatitis SCORAD and the subjects SCORAD score is reduced from baseline.

In one embodiment the invention relates to the IL-22R binding protein for use, the method or the use as above, wherein Atopic Dermatitis Disease Severity Outcome Measure is affected Body Surface Area (BSA) and the subjects BSA score is reduced from baseline.

In one embodiment the invention relates to the IL-22R binding protein for use, the method or the use as above, wherein Atopic Dermatitis Disease Severity Outcome Measure is a Patient reported outcome DLQI, POEM, EQ-5D-5L index, EQ-5D-5L VAS, WPAI-SHP, HADS, SCORAD Part C (pruritus VAS), ADSD Worst Itch and ADSD worst skin pain and the patient reported outcome is improved from baseline.

In one embodiment the invention relates to the IL-22R binding protein for use, the method or the use as above, wherein each dose of the IL-22R binding protein is administered as a pharmaceutical composition comprising 150±15 mg/mL-225 mg/ml±25 mg/ml of IL-22R binding protein, one or more disaccharides, one or more amino acids, optionally a surfactant and at a pH of 5.5-6.5 and with an osmolality of 280-450 mOsm/kg.

In one embodiment the invention relates to the IL-22R binding protein for use, the method or the use as above, wherein each dose of the IL-22R binding protein is administered as a pharmaceutical composition comprising about 150 mg/ml of the IL-22R binding protein, about 20 mM Histidine, about 80 mM Glycine, about 20 mM Methionine, about 180 mM Trehalose, optionally 20 mg/ml Tween 20 at a pH of 6.3 and with an Osmolality of 331 (mOsm/Kg).

In one embodiment the invention relates to the IL-22R binding protein for use, the method or the use as above, wherein the IL-22R binding protein is administered as a monotherapy.

In one embodiment the invention relates to the IL-22R binding protein for use, the method or the use as above, wherein the IL-22R binding protein is administered in combination with a second therapeutic agent selected from the group consisting of a topical corticosteroid, a topical calcineurin inhibitor, an anti-histamine, an emollient, or an anti-bacterial therapeutic.

In one embodiment the invention relates to the IL-22R binding protein for use, the method or the as above, wherein the subjects Atopic Dermatitis Disease Severity Outcome Measure is improved compared to baseline.

In some embodiments, the Atopic Dermatitis Disease Severity Outcome Measure is Eczema Area and Severity Index (EASI) and EASI score is reduced from baseline.

In some embodiments as above, the subject achieves a 50% or greater improvement in EASI score compared to baseline. In some embodiments the subject achieves a 50% or greater improvement in EASI score at week 16.

In some embodiments as above, the subject achieves a 75% or greater improvement in EASI score compared to baseline. In some embodiments the subject achieves a 75% or greater improvement in EASI score at week 16.

In some embodiments as above, the subject achieves a 90% or greater improvement in EASI score compared to baseline. In some embodiments the subject achieves a 90% or greater improvement in EASI score at week 16.

In some embodiments as above, the subject achieves a 100% improvement in EASI score compared to baseline. In some embodiments the subject achieves a 100% improvement in EASI score week 16.

In some embodiments as above, the subject achieves a 50% or greater improvement in EASI score compared to baseline. In some embodiments the subject achieves a 50% or greater improvement in EASI score at week 8.

In some embodiments as above, the subject achieves a 75% or greater improvement in EASI score compared to baseline. In some embodiments the subject achieves a 75% or greater improvement in EASI score at week 8.

In some embodiments as above, the subject achieves a 90% or greater improvement in EASI score compared to baseline. In some embodiments the subject achieves a 90% or greater improvement in EASI score at week 8.

In some embodiments as above, the subject achieves a 100% improvement in EASI score compared to baseline. In some embodiments the subject achieves a 100% improvement in EASI score week 8.

In some embodiments as above, the subject achieves a 50% or greater improvement in EASI score compared to baseline. In some embodiments the subject achieves a 50% or greater improvement in EASI score at week 6.

In some embodiments as above, the subject achieves a 75% or greater improvement in EASI score compared to baseline. In some embodiments the subject achieves a 75% or greater improvement in EASI score at week 6.

In some embodiments as above, the subject achieves a 90% or greater improvement in EASI score compared to baseline. In some embodiments the subject achieves a 90% or greater improvement in EASI score at week 6.

In some embodiments as above, the subject achieves a 100% improvement in EASI score compared to baseline. In some embodiments the subject achieves a 100% improvement in EASI score week 6.

In some embodiments as above, the Atopic Dermatitis Disease Severity Outcome Measure is the Validated Investigator Global Assessment Scale for Atopic Dermatitis (vIGA-AD) and the patients vIGA-AD score is reduced from baseline.

In some embodiments as above, the subject achieves an vIGA-AD score of 0 (clear) or 1 (almost clear). In some embodiments as above, the subject achieves an vIGA-AD score of 0 (clear) or 1 (almost clear) at week 16 or earlier such as week, 4, week 6, week 8 or week 12.

In some embodiments as above, the Atopic Dermatitis Disease Severity Outcome Measure is Severity Scoring of Atopic Dermatitis SCORAD and the subjects SCORAD score is reduced from baseline.

In some embodiments as above, the subject achieves a 25% or greater improvement in SCORAD score compared to baseline. In some embodiments the subject achieves a 25% or greater improvement in SCORAD score at week 16.

In some embodiments as above, the subject achieves a 50% or greater improvement in SCORAD score compared to baseline. In some embodiments the subject achieves a 50% or greater improvement in SCORAD score at week 16.

In some embodiments as above, the subject achieves a 75% or greater improvement in SCORAD score compared to baseline. In some embodiments the subject achieves a 75% or greater improvement in SCORAD score at week 16.

In some embodiments as above, the subject achieves a 90% or greater improvement in SCORAD score compared to baseline. In some embodiments the subject achieves a 90% or greater improvement in SCORAD score at week 16.

In some embodiments as above, the subject achieves a 100% improvement in SCORAD score compared to baseline. In some embodiments the subject achieves a 100% improvement in SCORAD score at week 16.

In some embodiments as above, the Atopic Dermatitis Disease Severity Outcome Measure is affected Body Surface Area (BSA) and the subjects BSA score is reduced compared to baseline.

In some embodiments as above, the subject achieves a 25% or greater improvement in BSA score compared to baseline. In some embodiments the subject achieves a 25% or greater improvement in BSA score at week 16.

In some embodiments as above, the subject achieves a 50% or greater improvement in BSA score compared to baseline. In some embodiments the subject achieves a 50% or greater improvement in BSA score at week 16.

In some embodiments as above, the subject achieves a 75% or greater improvement in BSA score compared to baseline. In some embodiments the subject achieves a 75% or greater improvement in BSA score at week 16.

In some embodiments as above, the subject achieves a 90% or greater improvement in BSA score compared to baseline. In some embodiments the subject achieves a 90% or greater improvement in BSA score at week 16.

In some embodiments as above, the subject achieves a 100% improvement in BSA score compared to baseline. In some embodiments the subject achieves a 100% improvement in BSA score at week 16.

In some embodiments as above, the Atopic Dermatitis Disease Severity Outcome Measure is a Patient reported outcome DLQI, POEM, EQ-5D-5L index, EQ-5D-5L VAS, WPAI-SHP, HADS, SCORAD Part C (pruritus VAS). ADSD Worst Itch and ADSD worst skin pain and the patient reported outcome is reduced from baseline.

In some embodiments as above, the subject achieves a 25% or greater improvement in DLQI score compared to baseline. In some embodiments the subject achieves a 25% or greater improvement in DLQI score at week 16.

In some embodiments as above, the subject achieves a 50% or greater improvement in DLQI score compared to baseline. In some embodiments the subject achieves a 50% or greater improvement in DLQI score at week 16.

In some embodiments as above, the subject achieves a 75% or greater improvement in DLQI score compared to baseline. In some embodiments the subject achieves a 75% or greater improvement in DLQI score at week 16.

In some embodiments as above, the subject achieves a 90% or greater improvement in DLQI score compared to baseline. In some embodiments the subject achieves a 90% or greater improvement in DLQI score at week 16.

In some embodiments as above, the subject achieves a 100% improvement in DLQI score compared to baseline. In some embodiments the subject achieves a 100% improvement in DLQI score at week 16.

In some embodiments as above, the subject has a DLQI score≥4 at baseline and achieves a 25%, 50%, 75% or greater improvement in DLQI score at week 16 compared to baseline.

In some embodiments as above, the subject has a DLQI score≥4 at baseline and achieves a 25%, 50%, 75% or greater improvement in DLQI score at week 32 compared to baseline.

In some embodiments as above, the subject achieves a 25% or greater improvement in POEM score compared to baseline. In some embodiments the subject achieves a 25% or greater improvement in POEM score at week 16

In some embodiments as above, the subject achieves a 50% or greater improvement in POEM score compared to baseline. In some embodiments the subject achieves a 50% or greater improvement in POEM score at week 16.

In some embodiments as above, the subject achieves a 75% or greater improvement in POEM score compared to baseline. In some embodiments the subject achieves a 75% or greater improvement in POEM score at week 16.

In some embodiments as above, the subject achieves a 90% or greater improvement in POEM score compared to baseline. In some embodiments the subject achieves a 90% or greater improvement in POEM score at week 16.

In some embodiments as above, the subject achieves a 100% improvement in POEM score compared to baseline. In some embodiments the subject achieves a 100% improvement in POEM score at week 16.

In some embodiments as above, the subject has a POEM score≥4 at baseline and achieves a 25%, 50%, 75% or greater improvement in POEM score at week 16 compared to baseline.

In some embodiments as above, the subject has a POEM score≥4 at baseline and achieves a 25%, 50%, 75% or greater improvement in POEM score at week 32 compared to baseline.

In some embodiments as above, the subject achieves a 25% or greater improvement in EQ-5D-5L INDEX score compared to baseline. In some embodiments the subject achieves a 25% or greater improvement in EQ-5D-5L INDEX score at week 16.

In some embodiments as above, the subject achieves a 50% or greater improvement in EQ-5D-5L INDEX score compared to baseline. In some embodiments the subject achieves a 50% or greater improvement in EQ-5D-5L INDEX score at week 16.

In some embodiments as above, the subject achieves a 75% or greater improvement in EQ-5D-5L INDEX score compared to baseline. In some embodiments the subject achieves a 75% or greater improvement in EQ-5D-5L INDEX score at week 16.

In some embodiments as above, the subject achieves a 90% or greater improvement in EQ-5D-5L INDEX score compared to baseline. In some embodiments the subject achieves a 90% or greater improvement in EQ-5D-5L INDEX score at week 16.

In some embodiments as above, the subject achieves a 100% improvement in EQ-5D-5L INDEX score compared to baseline. In some embodiments the subject achieves a 100% improvement in EQ-5D-5L INDEX score at week 16.

In some embodiments as above, the subject achieves a 25% or greater improvement in EQ-5D-5L VAS score compared to baseline. In some embodiments the subject achieves a 25% or greater improvement in EQ-5D-5L VAS score at week 16.

In some embodiments as above, the subject achieves a 50% or greater improvement in EQ-5D-5L VAS score compared to baseline. In some embodiments the subject achieves a 50% or greater improvement in EQ-5D-5L VAS score at week 16.

In some embodiments as above, the subject achieves a 75% or greater improvement in EQ-5D-5L VAS score compared to baseline. In some embodiments the subject achieves a 75% or greater improvement in EQ-5D-5L VAS score at week 16.

In some embodiments as above, the subject achieves a 90% or greater improvement in EQ-5D-5L VAS score compared to baseline. In some embodiments the subject achieves a 90% or greater improvement in EQ-5D-5L VAS score at week 16.

In some embodiments as above, the subject achieves a 100% improvement in EQ-5D-5L VAS score compared to baseline. In some embodiments the subject achieves a 100% improvement in EQ-5D-5L VAS score at week 16.

In some embodiments as above, the subject achieves a 25% or greater improvement in WPAI-SHP domain compared to baseline. In some embodiments the subject achieves a 25% or greater improvement in WPAI-SHP domain at week 16.

In some embodiments as above, the subject achieves a 50% or greater improvement in

WPAI-SHP domain compared to baseline. In some embodiments the subject achieves a 50% or greater improvement in WPAI-SHP domain at week 16.

In some embodiments as above, the subject achieves a 75% or greater improvement in WPAI-SHP domain compared to baseline. In some embodiments the subject achieves a 75% or greater improvement in WPAI-SHP domain at week 16.

In some embodiments as above, the subject achieves a 90% or greater improvement in WPAI-SHP domain compared to baseline. In some embodiments the subject achieves 90% or greater improvement in WPAI-SHP domain at week 16.

In some embodiments as above, the subject achieves a 100% improvement in WPAI-SHP domain compared to baseline. In some embodiments the subject achieves 100% improvement in WPAI-SHP domain week 16.

In some embodiments as above, the subject achieves a 25% or greater improvement in HADS score compared to baseline. In some embodiments the subject achieves a 25% or greater improvement in HADS score at week 16.

In some embodiments as above, the subject achieves a 50% or greater improvement in HADS score compared to baseline. In some embodiments the subject achieves a 50% or greater improvement in HADS score at week 16.

In some embodiments as above, the subject achieves a 75% or greater improvement in HADS score compared to baseline. In some embodiments the subject achieves a 75% or greater improvement in HADS score at week 16.

In some embodiments as above, the subject achieves a 90% or greater improvement in HADS score compared to baseline. In some embodiments the subject achieves a 90% or greater improvement in HADS score at week 16.

In some embodiments as above, the subject achieves a 100% improvement in HADS score compared to baseline. In some embodiments the subject achieves a 100% improvement in HADS score at week 16.

In some embodiments as above, the subject achieves a 25% or greater improvement in SCORAD Part c (pruritus vas) score compared to baseline. In some embodiments the subject achieves a 25% or greater improvement in SCORAD Part c (pruritus vas) score at week 16.

In some embodiments as above, the subject achieves a 50% or greater improvement in SCORAD Part c (pruritus vas) score compared to baseline. In some embodiments the subject achieves a 50% or greater improvement in SCORAD Part c (pruritus vas) score at week 16.

In some embodiments as above, the subject achieves a 75% or greater improvement in SCORAD Part c (pruritus vas) score compared to baseline. In some embodiments the subject achieves a 75% or greater improvement in SCORAD Part c (pruritus vas) score at week 16.

In some embodiments as above, the subject achieves a 90% or greater improvement in SCORAD Part c (pruritus vas) score compared to baseline. In some embodiments the subject achieves a 90% or greater improvement in SCORAD Part c (pruritus vas) score at week 16.

In some embodiments as above, the subject achieves a 100% improvement in SCORAD Part c (pruritus vas) score compared to baseline. In some embodiments the subject achieves a 100% improvement in SCORAD Part c (pruritus vas) score at week 16.

In some embodiments as above, the subject achieves a 25% or greater improvement in ADSD worst itch score compared to baseline. In some embodiments the subject achieves a 25% or greater improvement in ADSD worst itch score at week 16.

In some embodiments as above, the subject achieves a 50% or greater improvement in ADSD worst itch score compared to baseline. In some embodiments the subject achieves a 50% or greater improvement in ADSD worst itch score at week 16.

In some embodiments as above, the subject achieves a 75% or greater improvement in ADSD worst itch score compared to baseline. In some embodiments the subject achieves a 75% or greater improvement in ADSD worst itch score at week 16.

In some embodiments as above, the subject achieves a 90% or greater improvement in ADSD worst itch score compared to baseline. In some embodiments the subject achieves a 90% or greater improvement in ADSD worst itch score at week 16.

In some embodiments as above, the subject achieves a 100% improvement in ADSD worst itch score compared to baseline. In some embodiments the subject achieves a 100% improvement in ADSD worst itch score at week 16.

In some embodiments as above, the subject achieves a 25% or greater improvement in

ADSD worst skin pain score compared to baseline. In some embodiments the subject achieves a 25% or greater improvement in ADSD worst skin pain score at week 16.

In some embodiments as above, the subject achieves a 50% or greater improvement in ADSD worst skin pain score compared to baseline. In some embodiments the subject achieves a 50% or greater improvement in ADSD worst skin pain score at week 16.

In some embodiments as above, the subject achieves a 75% or greater improvement in ADSD worst skin pain score compared to baseline. In some embodiments the subject achieves a 75% or greater improvement in ADSD worst skin pain score at week 16.

In some embodiments as above, the subject achieves a 100% improvement in ADSD worst skin pain score compared to baseline. In some embodiments the subject achieves a 100% improvement in ADSD worst skin pain score at week 16.

In some embodiments as above, the subject has a ADSD worst skin pain score≥4 at baseline and achieves a 25%, 50%, 75% or greater improvement in ADSD worst skin pain score at week 16.

In some embodiments as above, the Atopic Dermatitis Disease Severity Outcome Measure is a change in difficulty falling asleep (weekly average), a change in nocturnal scratching (weekly average) or a change in frequency of waking up during the night (weekly average) compared to baseline

In some embodiments as above, the subject achieves a 25% or greater improvement in difficulty falling asleep (weekly average) compared to baseline. In some embodiments the subject achieves a 25% or greater improvement in difficulty falling asleep (weekly average) at week 16.

In some embodiments as above, the subject achieves a 50% or greater improvement in difficulty falling asleep (weekly average) compared to baseline. In some embodiments the subject achieves a 50% or greater improvement in difficulty falling asleep (weekly average) at week 16.

In some embodiments as above, the subject achieves a 75% or greater improvement in difficulty falling asleep (weekly average) compared to baseline. In some embodiments the subject achieves a 75% or greater improvement in difficulty falling asleep (weekly average) at week 16.

In some embodiments as above, the subject achieves a 100% improvement in difficulty falling asleep (weekly average) compared to baseline. In some embodiments the subject achieves a 100% improvement in difficulty falling asleep (weekly average) at week 16.

In some embodiments as above, the subject achieves a 25% or greater improvement in nocturnal scratching (weekly average) compared to baseline. In some embodiments the subject achieves a 25% or greater improvement in nocturnal scratching (weekly average) at week 16.

In some embodiments as above, the subject achieves a 50% or greater improvement in nocturnal scratching (weekly average) compared to baseline. In some embodiments the subject achieves a 50% or greater improvement in nocturnal scratching (weekly average) at week 16.

In some embodiments as above, the subject achieves a 75% or greater improvement in nocturnal scratching (weekly average) compared to baseline. In some embodiments the subject achieves a 75% or greater improvement in nocturnal scratching (weekly average) at week 16.

In some embodiments as above, the subject achieves a 100% improvement in nocturnal scratching (weekly average) compared to baseline. In some embodiments the subject achieves a 100% improvement in nocturnal scratching (weekly average) at week 16.

In some embodiments as above, the subject achieves a 25% or greater improvement in frequency of waking up during night (weekly average) compared to baseline. In some embodiments the subject achieves a 25% or greater improvement in frequency of waking up during night (weekly average) at week 16.

In some embodiments as above, the subject achieves a 50% or greater improvement in frequency of waking up during night (weekly average) compared to baseline. In some embodiments the subject achieves a 50% or greater improvement in frequency of waking up during night (weekly average) at week 16.

In some embodiments as above, the subject achieves a 75% or greater improvement in frequency of waking up during night (weekly average) compared to baseline. In some embodiments the subject achieves a 75% or greater improvement in frequency of waking up during night (weekly average) at week 16.

In some embodiments as above, the subject achieves a 100% improvement in frequency of waking up during night (weekly average) compared to baseline. In some embodiments the subject achieves a 100% improvement in frequency of waking up during night (weekly average) at week 16.

In some embodiments as above, the subject achieves a reduction in abundance of Staphylococcus aureus from baseline.

In some embodiments as above, the subject achieves a 25% or greater reduction in abundance of Staphylococcus aureus compared to baseline. In some embodiments the subject achieves a 25% or greater reduction in abundance of Staphylococcus aureus at week 16.

In some embodiments as above, the subject achieves a 50% or greater reduction in abundance of Staphylococcus aureus compared to baseline. In some embodiments the subject achieves a 50% or greater reduction in abundance of Staphylococcus aureus at week 16.

In some embodiments as above, the subject achieves a 75% or greater reduction in abundance of Staphylococcus aureus compared to baseline. In some embodiments the subject achieves a 75% or greater reduction in abundance of Staphylococcus aureus at week 16.

In some embodiments as above, the subject achieves a 100% reduction in abundance of Staphylococcus aureus compared to baseline. In some embodiments the subject achieves a 100% reduction in abundance of Staphylococcus aureus at week 16.

In some embodiments as above, the subject achieves a 25%, 50%, 75% or 100% reduction in abundance of Staphylococcus aureus compared to baseline. In some embodiments the subject achieves a 25%, 50%, 75% or 100% reduction in abundance of Staphylococcus aureus at week 32.

In some embodiments as above, the patient achieves a 50% or greater improvement in EASI score at week 32.

In some embodiments as above, the patient achieves a 75% or greater improvement in EASI score at week 32.

In some embodiments as above, the patient achieves a 90% or greater improvement in EASI score at week 32.

In some embodiments as above, the patient achieves a 100% improvement in EASI score at week 32.

In some embodiments as above, the patient achieves a 75% or greater improvement in EASI score at week 48.

In some embodiments as above, the patient achieves a vIGA-AD score of 0 (clear) or 1 (almost clear) at week 32.

In some embodiments as above, the patient achieves a vIGA-AD score of 0 (clear) or 1 (almost clear) at week 48.

In some embodiments as above, the patient achieves a 25% or greater improvement in SCORAD score at week 32.

In some embodiments as above, the patient achieves a 50% or greater improvement in SCORAD score at week 32.

In some embodiments as above, the patient achieves a 75% or greater improvement in BSA score at week 32.

In some embodiments as above, the patient achieves a 100% improvement in BSA score at week 32.

In some embodiments as above, the patient achieves a 25% or greater improvement in BSA score at week 32.

In some embodiments as above, the patient achieves a 50% or greater improvement in BSA score at week 32.

In some embodiments as above, the patient achieves a 75% or greater improvement in BSA score at week 32.

In some embodiments as above, the patient achieves a 100% improvement in BSA score at week 32.

In some embodiments as above, the patient achieves a 25% or greater improvement in DLQL score at week 32.

In some embodiments as above, the patient achieves a 50% or greater improvement in DLQL score at week 32.

In some embodiments as above, the patient achieves a 75% or greater improvement in DLQL score at week 32.

In some embodiments as above, the patient achieves a 100% improvement in DLQL score at week 32.

In some embodiments as above, the patient achieves a 25% or greater improvement in POEM score at week 32.

In some embodiments as above, the patient achieves a 50% or greater improvement in POEM score at week 32.

In some embodiments as above, the patient achieves a 75% or greater improvement in POEM score at week 32.

In some embodiments as above, the patient achieves a 100% improvement in POEM score at week 32.

In some embodiments as above, the patient achieves a 25% or greater improvement in EQ-5D-5L index score at week 32.

In some embodiments as above, the patient achieves a 50% or greater improvement in EQ-5D-5L index score at week 32.

In some embodiments as above, the patient achieves a 75% or greater improvement in EQ-5D-5L index score at week 32.

In some embodiments as above, the patient achieves a 100% improvement in EQ-5D-5L index score at week 32.

In some embodiments as above, the patient achieves a 25% or greater improvement in EQ-5D-5L VAS score at week 32.

In some embodiments as above, the patient achieves a 50% or greater improvement in EQ-5D-5L VAS score at week 32.

In some embodiments as above, the patient achieves a 75% or greater improvement in EQ-5D-5L VAS score at week 32.

In some embodiments as above, the patient achieves a 100% improvement in EQ-5D-5L VAS score at week 32.

In some embodiments as above, the patient achieves a 25% or greater improvement in WPAI-SHP domain at week 32.

In some embodiments as above, the patient achieves a 50% or greater improvement in WPAI-SHP domain at week 32.

In some embodiments as above, the patient achieves a 75% or greater improvement in WPAI-SHP domain at week 32.

In some embodiments as above, the patient achieves a 100% improvement in WPAI-SHP domain at week 32.

In some embodiments as above, the patient achieves a 25% or greater improvement in HADS score at week 32.

In some embodiments as above, the patient achieves a 50% or greater improvement in HADS score at week 32.

In some embodiments as above, the patient achieves a 75% or greater improvement in HADS score at week 32.

In some embodiments as above, the patient achieves a 100% improvement in HADS score at week 32.

In some embodiments as above, the patient achieves a 25% or greater improvement in SCORAD Part C (VAS pruritus) score at week 32.

In some embodiments as above, the patient achieves a 50% or greater improvement in SCORAD Part C (VAS pruritus) score at week 32.

In some embodiments as above, the patient achieves a 75% or greater improvement in

SCORAD Part C (VAS pruritus) score at week 32.

In some embodiments as above, the patient achieves a 100% improvement in SCORAD Part C (VAS pruritus) score at week 32.

In some embodiments as above, the patient achieves a 25% or greater improvement in ADSD worst Itch score at week 32.

In some embodiments as above, the patient achieves a 50% or greater improvement in ADSD worst Itch score at week 32.

In some embodiments as above, the patient achieves a 75% or greater improvement in ADSD worst Itch score at week 32.

In some embodiments as above, the patient achieves a 100% improvement in ADSD worst Itch score at week 32.

In some embodiments as above, the patient achieves a 25% or greater improvement in ADSD worst skin pain score at week 32.

In some embodiments as above, the patient achieves a 50% or greater improvement in ADSD worst skin pain score at week 32.

In some embodiments as above, the patient achieves a 75% or greater improvement in ADSD worst skin pain score at week 32.

In some embodiments as above, the patient achieves a 100% improvement in ADSD worst skin pain score at week 32.

In some embodiments as above, the patient achieves a 25% or greater improvement in difficulty falling asleep (weekly average) at week 32.

In some embodiments as above, the patient achieves a 50% or greater improvement in difficulty falling asleep (weekly average) at week 32.

In some embodiments as above, the patient achieves a 75% or greater improvement in difficulty falling asleep (weekly average) at week 32.

In some embodiments as above, the patient achieves a 100% improvement in difficulty falling asleep (weekly average) at week 32.

In some embodiments as above, the patient achieves a 25% or greater improvement in nocturnal scratching (weekly average) at week 32.

In some embodiments as above, the patient achieves a 50% or greater improvement in nocturnal scratching (weekly average) at week 32.

In some embodiments as above, the patient achieves a 75% or greater improvement in nocturnal scratching (weekly average) at week 32.

In some embodiments as above, the patient achieves a 100% improvement in nocturnal scratching (weekly average) at week 32.

In some embodiments as above, the patient achieves a 25% or greater improvement in in frequency of waking up during the night (weekly average) at week 32.

In some embodiments as above, the patient achieves a 50% or greater improvement in in frequency of waking up during the night (weekly average) at week 32.

In some embodiments as above, the patient achieves a 75% or greater improvement in in frequency of waking up during the night (weekly average) at week 32.

In some embodiments as above, the patient achieves a 100% improvement in in frequency of waking up during the night (weekly average) at week 32.

In some embodiments as above, the subject has a BSA score of 20% or lower at baseline

In some embodiments as above, the subject has a BSA score of 15% or greater at baseline.

In some embodiments as above, the subject has a BSA score greater than 20% at baseline.

In some embodiments as above, the subject is an individual aged 12 years or older.

In some embodiments as above, the subject is an individual aged 50 years or younger.

In some embodiments as above, the subject is an individual aged 12 years to 50 years.

In some embodiments as above, the subject is an adult.

In some embodiments as above, the subject is an adolescent.

### Administration

In the methods described herein, the IL-22R binding protein (e.g. an anti-IL-22R antibody or an IL-22R-binding fragment thereof) may be administered by any appropriate method. Typically, administration is parenteral, e.g. intradermal, intramuscular, intravenous and subcutaneous. Subcutaneous administration is particularly preferred (e.g. as illustrated in the examples). Each dose of the IL-22R binding protein may therefore be administered subcutaneously.

Administration is preferably in a “therapeutically effective amount”, this being sufficient to show improvement or maintained improvement in one or more AD-associated parameter or patient-related outcome as described herein or Atopic Dermatitis Disease Severity Outcome Measure as described herein, or achievement of a low disease state.

Administration may be by any convenient route, for example by infusion or bolus injection, by absorption through epithelial or mucocutaneous linings (e.g. oral mucosa, rectal and intestinal mucosa, etc.).

Subcutaneous or intravenous delivery may be with a standard needle and syringe (e.g. including with a prefilled syringe). It is envisaged that the methods described herein will not be restricted to use in the clinic. Therefore, subcutaneous injection using a needle free device is also preferred. Such delivery devices can be reusable or disposable. Numerous reusable pen and autoinjector delivery devices are known in the art and may find use in the present invention. Examples include AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ 1, 11 and 111 (Nova Nordisk, Copenhagen, Denmark), NOVOPEN JUNIOR™ (Nova Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany). Exemplary disposable pen delivery devices for subcutaneous delivery that may find use in the present invention applications include the SOLOSTART pen (Sanofi-Aventis), the FLEXPEN™ (Nova Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park Ill.).

Each dose of IL-22R binding protein of is not necessarily administered in a single administration step (e.g. one injection Indeed, depending on the concentration of the IL-22R binding protein (e.g. in the pharmaceutical composition), one, two, three or more administration steps (e.g. one, two, three or more injections) may be required to provide the subject with the required amount IL-22R binding protein (e.g. a 450 mg dose, for example). Thus, in some embodiments, each dose of the IL-22R binding protein is administered in one two, or three injections (e.g. subcutaneously). Typically subcutaneous injections have a volume of around 1.5 mL or less, such as a volume of from 0.2 to 1.5 mL.

### Monotherapy and Combination Therapy

The methods described herein may be a monotherapy. As used herein, the term “monotherapy” is a therapy which uses a single drug to treat a disease or condition. Therefore, a subject that is treated with a monotherapy will receive only a single drug to treat the relevant disorder, e.g. AD, a skin infection, pruritus or eczema-related sleep interference. For example, an anti-IL-22R antibody monotherapy refers to a monotherapy which comprises the administration of anti-IL-22R antibody to the subject as the sole drug for the treatment of AD or a skin infection.

The methods described herein may be a combination therapy. As used herein, the term “combination therapy” is a therapy which uses more than one drug to treat a disease or condition. For example, a subject that is treated with a combination therapy will receive more than one drug (e.g. two, three or more) to treat AD.

In some embodiments, the IL-22R binding protein is administered in combination with a topical therapy (such as a topical corticosteroid or a topical calcineurin inhibitor). In some instances, the additional treatment (e.g. TCS or TCI) is administered as needed by the subject.

In some cases, the IL-22R binding protein is administered in combination with a second therapeutic agent selected from the group consisting of a topical corticosteroid, a topical calcineurin inhibitor, an anti-histamine, an emollient, or an anti-bacterial therapeutic. In some cases, the IL-22R binding protein is administered in combination with a Group I, Group II, Group III or Group IV corticosteroid. Preferably, the IL-22R binding protein can be administered in combination with mometasone furoate (e.g. 0.1% cream).

### Pharmaceutical Compositions and Formulations

The present invention envisages methods where each dose of the IL-22R binding protein (e.g. an anti-IL-22R antibody or an IL-22R-binding fragment thereof) is administered as a pharmaceutical composition.

The pharmaceutical compositions may be formulated with suitable carriers, excipients, and other agents that provide suitable transfer, delivery, tolerance, and the like. A multitude of formulations can be found in the formulary known to all pharmaceutical chemists: Remington's Pharmaceutical Sciences, Mack Publishing Company, Easton, PA. The dose administered to a patient according to the methods described herein may be varied depending upon the age and the size of the patient, symptoms, conditions, route of administration, and the like. The dose can be calculated according to body weight or body surface area.

Thus, the pharmaceutical compositions may comprise, in addition to the active ingredient (i.e. the IL-22R binding protein), a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous or subcutaneous. Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. For intravenous injection or subcutaneous injection, the pharmaceutical composition may be a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The pharmaceutical composition may be a liquid formulation or a lyophilized formulation which is reconstituted before use. As excipients for a lyophilized formulation, for example, sugar alcohols, or saccharides (e.g. mannitol or glucose) may be used. In the case of a liquid formulation, the pharmaceutical composition is usually provided in the form of containers with defined volume, including sealed and sterilized plastic or glass vials, ampoules and syringes, as well as in the form of large volume containers like bottles. Preferably, in the methods described herein, the pharmaceutical composition is a liquid formulation.

Preferably, the IL-22R binding protein may be present within the pharmaceutical composition at a concentration of from 1 mg/mL to 200 mg/mL, more preferably 150 mg/mL.

Preferably, the pharmaceutical composition may be buffered to a pH of 5.2 to 5.7, most preferably 5.5 (±0.1). The selection of such a pH confers significant stability to the pharmaceutical composition. Examples of alternative buffers that control the pH in this range include succinate, gluconate, histidine, citrate, phosphate, glutarate, cacodylate, sodium hydrogen maleate, tris(hydroxymethyl)aminomethane (Tris), 2-(N-morpholino) ethane-sulphonic acid (MES), imidazole. Preferably, the buffer is acetate buffer, more preferably sodium acetate buffer.

Preferably, the acetate buffer is present within the pharmaceutical composition in an amount of from 1 mM to 100 mM, more preferably from 30 mM to 70 mM, especially 50 mM. It will be appreciated that references to “pharmaceutically acceptable excipient” includes references to any excipient conventionally used in pharmaceutical compositions. Such excipients may typically include one or more surfactant, inorganic or organic salt, stabilizer, diluent, solubilizer, reducing agent, antioxidant, chelating agent, preservative and the like. Examples of a typical surfactant include: nonionic surfactants (HLB 6 to 18) such as sorbitan fatty acid esters (e.g. sorbitan monocaprylate, sorbitan monolaurate, sorbitan monopalmitate), glycerine fatty acid esters (e.g. glycerine monocaprylate, glycerine monomyristate, glycerine monostearate), polyglycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxyethylene polyoxypropylene alkyl ethers (e.g. polyoxyethylene polyoxypropylene glycol ether, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils (e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearyl amide); anionic surfactants such as C10-C18 alkyl sulfates salts (e.g. sodium cetyl sulfate, sodium lauryl sulfate, sodium oleyl sulfate), polyoxyethylene C10-C18 alkyl ether sulfates salts with an average of 2 to 4 moles of ethylene oxide (e.g. sodium polyoxyethylene lauryl sulfate), and C8-C18 alkyl sulfosuccinate ester salts (e.g. sodium lauryl sulfosuccinate ester); and natural surfactants such as lecithin, glycerophospholipid, sphingophospholipids (e.g. sphingomyelin), and sucrose esters of C12-C18 fatty acids. The surfactant may be selected from polyoxyethylene sorbitan fatty acid esters. Preferred surfactants are polysorbate 20, 21, 40, 60, 65, 80, 81 and 85, most preferably polysorbate 20 and 80, especially polysorbate 80.

Preferably, the surfactant is present within the pharmaceutical composition in an amount of from 0.001% to 0.1% (w/w), more preferably 0.005% and 0.05% (w/w), especially 0.01% (w/w).

Examples of a typical inorganic salt include: sodium chloride, potassium chloride, calcium chloride, sodium phosphate, sodium sulphate, ammonium sulphate, potassium phosphate and sodium bicarbonate or any other sodium, potassium or calcium salt. Preferably, the inorganic salt is sodium chloride.

Preferably, the inorganic salt is present within the pharmaceutical composition in an amount of from 10 mM to 200 mM, more preferably from 60 mM to 130 mM, especially 85 mM. Examples of a reducing agent include N-acetylcysteine, Nacetylhomocysteine, thioctic acid, thiodiglycol, thioethanolamine, thioglycerol, thiosorbitol, thioglycolic acid and a salt thereof, sodium thiosulfate, glutathione, and a CI-C7 thioalkanoic acid.

Examples of an antioxidant include erythorbic acid, dibutylhydroxytoluene, butylhydroxyanisole, alpha-tocopherol, tocopherol acetate, L-ascorbic acid and a salt thereof, L-ascorbic acid palmitate, L-ascorbic acid stearate, sodium bisulfite, sodium sulfite, triamyl gallate and propyl gallate.

Examples of a chelating agent include disodium ethylenediaminetetraacetate (EDTA), sodium pyrophosphate and sodium metaphosphate.

Examples of a stabiliser include creatinine, an amino acid selected from histidine, alanine, glutamic acid, glycine, leucine, phenylalanine, methionine, isoleucine, proline, aspartic acid, arginine, lysine and threonine, a carbohydrate selected from sucrose, trehalose, sorbitol, xylitol and mannose, surfactants selected from polyethylene glycol (PEG; e.g. PEG3350 or PEG 4000) or polyoxyethylene sorbitan fatty acid esters (e.g. polysorbate 20 or polysorbate 80), or any combination thereof.

In one preferred embodiment the stabiliser comprises a single carbohydrate (e.g. trehalose). In an alternatively preferred embodiment the stabilizer comprises an amino acid in combination with a carbohydrate (e.g. trehalose and alanine or trehalose, alanine and glycine).

In a further alternatively preferred embodiment the stabiliser comprises an amino acid in combination with a carbohydrate and a surfactant (e.g. trehalose, alanine and PEG3350; trehalose, proline and PEG3350; trehalose, alanine and polysorbate 80; trehalose, proline and polysorbate 80; trehalose, alanine, glycine and PEG3350; trehalose, alanine, glycine and polysorbate 80).

In a yet further alternatively preferred embodiment the stabiliser comprises an amino acid in combination with a surfactant (e.g. alanine and PEG3350 or alanine, glycine and PEG3350). In a yet further alternatively preferred embodiment the stabiliser comprises a carbohydrate in combination with a surfactant (e.g. trehalose and PEG3350 or trehalose and polysorbate 80).

Examples of a preservative include octadecyldimethylbenzyl ammonium chloride, hexamethonium chloride, benzalkonium chloride (a mixture of alkylbenzyldimethylammonium chlorides in which the alkyl groups are long chain compounds), benzethonium chloride, aromatic alcohols such as phenol, butyl and benzyl alcohol, alkyl parabens such as methyl or propyl paraben, catechol, resorcinol, cyclohexanol, 3-pentanol, and m-cresol. In a preferred embodiment, the pharmaceutical composition comprises an IL-22Rbinding protein as described herein, a surfactant and an inorganic salt buffered to a pH of 5.5±0.1 with acetate buffer.

Exemplary pharmaceutical compositions that can be used in the context of the present invention are disclosed in, for example EP22177269.2.

Suitable formulations is for example a formulation comprising
150±15 mg/mL-225 mg/mL±25 mg/ml of 230C9,
one or more disaccharides,
one or more amino acids,
optionally a surfactant,
at pH 5.5-6.5, having an osmolality of 280-450 mOsm/kg.

Suitably the formulation is selected from a formulation A containing about 150 mg/ml of 230C9, 20 mM Histidine, 80 mM Glycine, 20 mM Methionine, 180 mM Trehalose, optionally 20 mg/ml Tween 20 having a pH of 6.3 and an Osmolality of 331 (mOsm/Kg).

Another formulation is formulation B comprising A containing about 200 mg/ml of 230C9, 20 mM Histidine, 80 mM Glycine, 100 mM Trehalose, 0.2 mg/ml Tween 20, at a pH of 6 and an Osmolality of 358 (mOsm/Kg).

The following are examples of the practice of the invention. They are not to be construed as limiting the scope of the invention in any way.

### Example 1

A phase 2a, randomised, double-blind, placebo-controlled, multi-site, proof of concept trial to evaluate the efficacy and safety of 230C9 in adult subjects with moderate to severe atopic dermatitis (AD).

The primary objective was to compare the efficacy of 230C9 with placebo in subjects with moderate to severe AD. Primary endpoint: Change in Eczema Area and Severity Index (EASI) score from baseline to Week 16.

Secondary objective: To compare the safety of 230C9 with placebo in subjects with moderate to severe AD. Secondary endpoints: Number of treatment-emergent adverse events from baseline to Week 16 per subject.

A total of 52 subjects was randomised 1:1 to receive 230C9 450 mg Q2W (26 subjects) and placebo Q2W (26 subjects).

### Baseline Characteristics

TABLE 6

| | 230C9 450 mg | Placebo | Total |
|---|---|---|---|
| Randomised | 29 | 29 | 58 |
| Age (years, mean value) | 38.1 | 32.8 | 35.4 |
| Female | 58.6 | 55.2 | 56.9 |
| Weight (kg, mean value) | 74.47 | 75.29 | 74.88 |
| BM(kg/m², mean value | 25.93 | 25.66 | 25.79 |
| Duration of AD (years, mean | 26.9 | 23.4 | 25.1 |
| value) | | | |

| | N (%) | N (%) | N (%) |
|---|---|---|---|
| EASI score <21 | 14 (48.3) | 13 (44.8) | 27 (46.6) |
| EASI score ≥21 | 15 (51.7) | 16 (55.2) | 31 (53.4) |
| Moderate vIGA-ADscore | 17 (58.6) | 20 (69.0) | 37 (63.8) |
| Severe vIGA-AD score | 12 (41.4) | 9 (31.0) | 21 (36.2) |

| | Mean (SD) | Mean (SD) | Mean (SD) |
|---|---|---|---|
| EASI score | 26.93 (11.7) | 26.12 (9.80) | 26.53 (10.74) |
| Worst Daily Pruritus score | 7.57 (1.63) | 7.53 (1.56) | 7.55 (1.58) |
| POEM score | 22.4 (4.7) | 19.2 (5.4) | 20.8 (5.3) |
| DLQL | 17.1 (7.7) | 14.0 (6.0) | 15.6 (7.0) |
| BSA | 40.2 (21.4) | 38.6 (18.5) | 39.4 (19.9) |

| | N (%) | N (%) | N (%) |
|---|---|---|---|
| Systemic steroids | 17 (58.6) | 12 (41.4) | 29 (50.0%) |
| Topical corticosteroids | 28 (96.6) | 29 (100.0) | 57 (98.3) |
| Topical calcineurin inhibitors | 19 (65.5) | 19 (65.5) | 38 (65.5) |
| Dupilumab | 5 (17.2) | 4 (13.8) | 9 (15.5) |
| Cyclosporing | 7 (24.1) | 9 (31.0) | 16 (27.6) |
| Methotrexate | 1 (3.4) | | 1 (1.7) |
| Other immunosuppresant | 2 (6.9) | | 2 (3.4) |
| Phototherapy | 11 (37.9) | 11 (37.9) | 22 (37.9) |
| Other systemic medication | 4 (13.8) | 3 (10.3) | 7 (12.1) |

### Investigational Medicinal Product (IMP)

Lyophilised powder comprising 230C9 for reconstitution (using 1.0 ml sterilised water adding up to a volume of 1.2 ml/vial). Reconstituted formulation: 230C9 150 mg/ml, L-histidine 1.5 mg/ml, L-histidine HCl monohydrate 2.2.mg/ml, Sucrose 61.6 mg/ml, L-Arginine HCL 10.5 mg/ml, Polysorbate 20 0.4 mg/ml at pH 6.0.

Dose and frequency: 450 mg Q2W (3 injections of 150 mg 230C9)
Method of administration: Subcutaneous

### Trial Design

The trial consisted of a screening period of up to 4 weeks (Weeks −4 to −0) including an applicable washout period of 1 week (Week-1 to 0).

Subjects must stop treatment with TCS, TCI, topical PDE-4 inhibitors (PDE-4=phosphodiesterase-4; Q2W=every second week; TCI=topical calcineurin inhibitor(s); TCS=topical corticosteroid(s)), or other topical prescription treatments 1 week prior to randomization. All subjects must use an emollient twice daily (or more frequently, if needed) for at least 7 days before Day 1 (baseline) and throughout the treatment period (until Week 16).

In addition to the Q2W dosing schedule during the treatment phase, both treatment groups will receive an additional dose of their allocated treatment (450 mg 230C9 or placebo) at Visit 4 (Week 1).

The treatment period was 16 weeks (Weeks 0 to 16), followed by a 16-weeks off-treatment follow-up period for the assessment of safety and anti-drug antibodies (ADA) (Weeks 16 to 32).

After providing informed consent and successful completion of the screening and washout period, subjects were randomized in a 1:1 ratio to receive 450 mg 230C9 Q2W or placebo Q2W. Each subject received 3 SC injections (each 1.0 mL) of 150 mg 230C9 or placebo to receive a total dose of 450 mg 230C9 (3.0 mL) or placebo (3.0 mL). In addition to this Q2W dosing schedule, both treatment groups received an additional dose of their allocated treatment (230C9 450 mg or placebo) at Visit 4 (Week 1). Randomization was stratified by the disease severity at baseline; one stratum, ‘stratum A’, was defined as having EASI<21 and the other stratum, ‘stratum B’, was defined as having EASI≥21.

During the treatment period, final dosing was administered at Week 14, regardless of AD improvement. Rescue treatment: if medically necessary (i.e., due to worsening of AD or intolerable AD symptoms), rescue treatment for AD in the form of TCS/TCI could be used from Week 4 at the discretion of the investigator. The primary endpoint was be assessed at Week 16, and the final safety assessment will be conducted at Week 32.

Results: Efficacy at Week 16 230C9 demonstrated significant efficacy over vehicle. The proportion of patients who achieved an EASI-50, EASI-75, EASI-90, EASI-100 and vIGA-AD 0/1 at week 16 is shown in table 7.

TABLE 7
The primary analysis subjects who received rescue medication
or permanently discontinued 230C9 were considered non
responders. Subjects with missing data was imputed
as non-responders. A logistic regression model stratified
by region and baseline EASI score was used.

| | 230C9 | | | |
|---|---|---|---|---|
| | 450 mg, Q2W | Placebo | Difference | |
| | (N = 29) | (N029) | in % | |
| | Responder % | Responder % | (95% CI) | P-value |
| EASI-50 | 48.1 | 17.6 | 35.5 (8.5 | 0.007 |
| | | | to 52.5) | |
| EASI-75 | 41.6 | 3.7 | 27.9 (6.5, 49.3) | 0.011 |
| EASI-90 | 30.8 | 3.5 | 27.2 (9.4, 45.1) | 0.003 |
| EASI-100 | 20.9 | 0.0 | 20.9 (6.1, 35.7) | 0.006 |
| vIGA-AD 0/1 | 27.3 | 7.0 | 20.3 (1.4, 39.1) | 0.035 |

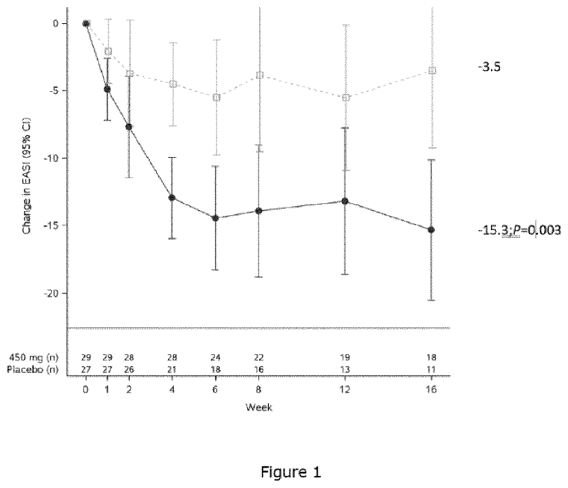

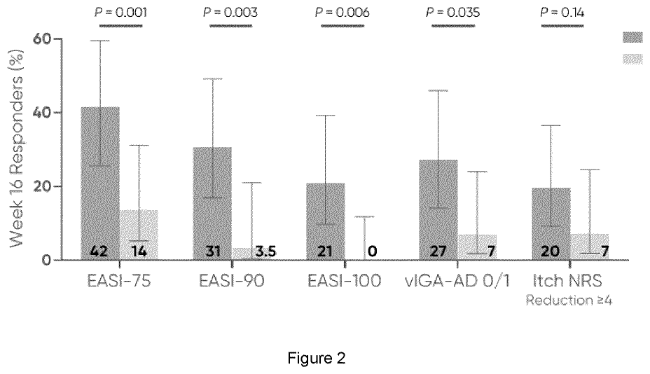

FIG. 1 show the change in EASI score of the first 16 weeks. FIG. 2 show the % responders at week 16.

### Results: Safety

All adverse invents were mild to moderate

TABLE 8

| | 230C9 450 mg Q2W | | | | Placebo | | | |
|---|---|---|---|---|---|---|---|---|
| | N | % | E | R | N | % | E | R |
| Adverse event | 19 | 65.5 | | 500.51 | 12 | 41.4 | 26 | 354.74 |
| Serious | | | | | | | | |
| Severe | | | | | 1 | 3.4 | 2 | 27.29 |
| Fatal | | | | | | | | |
| Probably related | 3 | 10.3 | 4 | 48.83 | 1 | 3.4 | 2 | 27.29 |
| Possibly related | 4 | 13.8 | 6 | 73.25 | 2 | 6.9 | 3 | 40.93 |
| Discont IMP | 6 | 20.7 | 6 | 73.25 | 2 | 6.9 | 2 | 27.29 |
| Withdrawn from | 2 | 6.9 | 2 | 24.42 | 1 | 3.4 | 1 | 13.64 |
| trial | | | | | | | | |

N = Number of subjects exposed. % = Percentage of subjects. E = Number of adverse events. R = Events/100 exposure years. IMP investigational medicinal product.

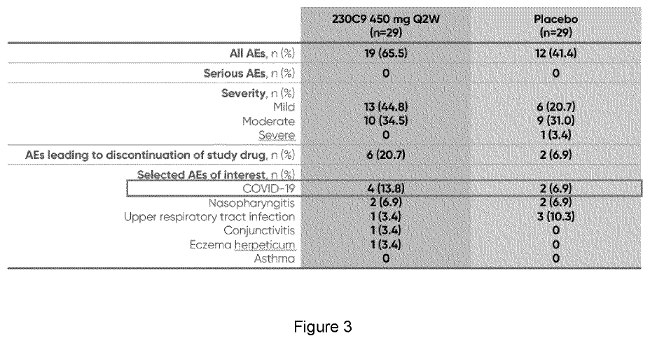

In FIG. 3 is an overall summary of AEs at week 16.

Discontinuation of IMP in active arm is mostly due to covid-19 infections (4/6), 1 due to eczema herpeticum, 1 due to dermatitis atopic.

### Example 2

A phase 2b, randomized, double-blind, placebo-controlled, multi-site, parallel-group, dose-finding trial to evaluate the efficacy and safety of different doses of subcutaneously (SC) administered 230C9 in adult subjects with moderate-to-severe atopic dermatitis (AD).

The primary objective is to compare the efficacy of 4 different dosage regimens of 230C9 with placebo in subjects with moderate-to-severe AD. Primary endpoint: Percent change in Eczema Area and Severity Index (EASI) score from baseline to Week 16.

The secondary objective is to compare the safety of 4 different doses or dose schedules of 230C9 with placebo in subjects with moderate-to-severe AD. Number of treatment-emergent adverse events (TEAEs) recorded for each subject from baseline to Week 16.

A total of approximately 250 subjects (at least 25 Japanese subjects) will randomised 1:1:1:1:1 to receive:

4 different 230C9 dose regimens and 1 placebo regimen of bi-weekly SC injections, each with a preceding loading dose regimen, will be tested:
Dose regimen 1: 230C9 600 mg SC Weeks 0, 1, 2, 3 and then 600 mg SC Q2W from Week 4 (50 subjects).
Dose regimen 2: 230C9 450 mg SC Weeks 0, 1, 2 and then 450 mg SC Q2W from Week 4 (50 subjects).
Dose regimen 3: 230C9 600 mg SC Weeks 0 and 2 and then 300 mg SC Q2W from Week 4 (50 subjects).
Dose regimen 4: 230C9 300 mg SC Weeks 0 and 2 and then 150 mg SC Q2W from Week 4 (50 subjects).

Placebo regimen: Placebo SC Weeks 0, 1, 2, 3 and then placebo SC Q2W from Week 4 (50 subjects).

### Investigational Medicinal Product (IMP)

IMP: 150 mg/ml 230C9, L-histidine 1.1 mg/ml, L-histidine HCL monohydrate 2.7 mg/ml, Glycine 6.0 mg/ml, Trehalose 68.1 mg/ml, Methionine 3.0 mg/ml, Polysorbate 20 0.2 mg/ml at pH 6.0.

| | 230C9, 150 mg/mL | Placebo |
|---|---|---|
| Dose regimen 1 | | |
| 230C9 600 mg | 2 × 2 mL | 1 × 1 mL |
| Dose regimen 2 | | |
| 230C9 450 mg | 1 × 2 mL | 1 × 2 mL |
| | 1 × 1 mL | |
| Dose regimen 3 | | |
| 230C9 600 mg (loading dose) | 2 × 2 mL | 1 × 1 mL |
| 230C9 300 mg | 1 × 2 mL | 1 × 2 mL |
| | | 1 × 1 mL |
| Dose regimen 4 | | |
| 230C9 300 mg (loading dose) | 1 × 2 mL | 1 × 2 mL |
| | | 1 × 1 mL |
| 230C9 150 mg | 1 × 1 mL | 2 × 2 mL |
| Placebo regimen | | |
| Placebo | — | 2 × 2 mL |
| | | 1 × 1 mL |

### Inclusion and Exclusion Criteria

### Inclusion Criteria

Subjects are Eligible to be Included in the Trial Only if all of the Following Criteria Apply:
1. Signed and dated informed consent has been obtained prior to any protocol-related procedures.
2. 18-75 years old (both included) at screening (Visit 1).
3. Willingness to comply with the clinical trial protocol.
4. At screening, diagnosis of AD as defined by the Hanifin and Rajka (1980) criteria for AD.
   History of AD for ≥1 year.
5. Subjects who have a recent history (within 12 months before screening) with documented inadequate response to treatment with TCS (±TCI as appropriate) or for whom these topical AD treatments are medically inadvisable (e.g. due to important side effects or safety risks*). *Important side effects or safety risks are those that outweigh the potential treatment benefits and include intolerance to treatment, hypersensitivity reactions, significant skin atrophy, and systemic effects, as assessed by the investigator, or by the subject's treating physician.
   Inadequate response is defined as failure to achieve and maintain remission or a low disease activity state (comparable to IGA 0=clear to 2=mild) despite treatment with a daily regimen of TCS of medium to higher potency (EU class II-IV, JP≥Medium, US class I-V) (±TCI as appropriate), applied for at least 28 days or for the maximum duration recommended by the product prescribing information (e.g. 14 days for super potent TCS), whichever is shorter.
   Subjects with documented systemic treatment or phototherapy for AD in the past 1 year are considered as inadequate responders to TCS treatment and are potentially eligible for trial inclusion after appropriate washout.
6. EASI score≥12 at screening and ≥16 at baseline.
7. vIGA-AD score≥3 at screening and baseline.
8. BSA of AD involvement≥10% at screening and baseline.
9. ADSD Worst Itch score (weekly average)≥4 at baseline. The baseline weekly average will be calculated from daily assessments of itch severity during the 7 days immediately preceding the baseline visit (Day-7 to Day-1). A minimum of 4 itch scores out of the 7 days is required to calculate the baseline average score.
10. The subject agrees to use moisturizers from screening throughout the trial.
11. A woman of childbearing potential* must use a highly effective ** form of birth control throughout the trial and for at least 18 weeks after last administration of IMP. * A woman of childbearing potential is defined as a female subject aged≥12 years or a younger girl who, at the discretion of the investigator, is deemed to be of reproductive potential. A woman is defined as not being of childbearing potential if she is postmenopausal (at least 12 months with no menses prior to screening without an alternative medical cause), or surgically sterile (hysterectomy, bilateral salpingectomy, or bilateral oophorectomy).** A highly effective method of birth control is defined as one which results in a low failure rate (less than 1% per year) such as bilateral tubal occlusion, intrauterine device (IUD), intrauterine hormone-releasing system (IUS), combined (oestrogen- and progestogen-containing) hormonal contraception associated with inhibition of ovulation (oral, intravaginal, transdermal), progestogen-only hormonal contraception associated with inhibition of ovulation (oral, injectable, implantable), sexual abstinence (when this is in line with the preferred and usual life style of the subject and not just being without a current partner), same-sex partner, or vasectomized partner (given that the subject is monogamous).

### Exclusion Criteria

Subjects are excluded from the trial if any of the following criteria apply:
1. Major* surgery within 8 weeks prior to screening, or planned inpatient surgery or hospitalization during the trial period (*at the discretion of the investigator).
2. Active dermatologic condition that could confound the diagnosis of AD or interfere with assessment of the treatment (e.g. scabies, contact dermatitis, rosacea, urticaria, or psoriasis).
3. History of cancer, with the following exceptions:
   Subjects who have had basal cell carcinoma, localized squamous cell carcinoma of the skin or in situ carcinoma of the cervix are eligible provided that the subject is in remission and curative therapy was completed at least 12 months prior to screening.
   Subjects who have had other malignancies are eligible provided that the subject is in remission and curative therapy was completed at least 5 years prior to screening.
4. History of or current immunodeficiency syndrome.
5. History of anaphylaxis following any biologic therapy.
6. History of clinically significant infection within 4 weeks prior to baseline which, in the opinion of the investigator, may compromise the safety of the subject in the trial, interfere with evaluation of the IMP, or reduce the subject's ability to participate in the trial. Clinically significant infections are defined as:
   a systemic infection.
   a serious skin infection requiring parenteral (IV or intramuscular) antibiotics, antiviral, or antifungal medication.
   NOTE: Subject may be rescreened (once) after infection resolves
7. Skin infection within 7 days prior to baseline. NOTE: Subject may be rescreened (once) after infection resolves.
8. Presence of hepatitis B or C infection at screening. These are defined as: 1) Positive hepatitis C Ab AND positive hepatitis C RNA, or 2) Positive HBsAg, or 3) Negative anti-HBs Ab AND positive anti-HBC Ab.
9. History of HIV infection or positive HIV serology at screening.
10. Evidence of active or latent tuberculosis according to local standard of care for patients requiring initiation of a biologic treatment.
   A tuberculosis test can be performed at the central or local laboratory.
11. ALT or AST level≥2.0 times the ULN at screening.
12. History of attempted suicide or is at significant risk of suicide (either in the opinion of the investigator or defined as a “yes” to suicidal ideation questions no. 4 or 5 or answering “yes” to suicidal behavior on the C-SSRS Screening version).
13. Known or suspected hypersensitivity to any component(s) of the IMP.
14. Any disorder* at screening and/or baseline, which is not stable in the opinion of the investigator, and could: *Examples include but are not limited to cardiovascular, gastrointestinal, hepatic, renal, neurological, musculoskeletal, infectious, endocrine, metabolic, hematological, immunological, and psychiatric disorders, and major physical impairment.
   Affect the safety of the subject throughout the trial.
   Influence the results of the trial.
   Impede the subject's ability to complete the trial.
15. Any significant abnormal finding* at screening and/or baseline which may in the opinion of the investigator: *The abnormal finding must be clinically significant and observed during the screening period. Examples include abnormal findings in physical examination, vital signs, ECG, hematology, biochemistry, or urinalysis.
   Put the subject at risk because of their participation in the trial.
   Influence the results of the trial.
   Influence the subject's ability to complete the trial.
16. Current or recent chronic alcohol or drug abuse, or any other condition associated with poor compliance as judged by the investigator.
17. Women who are pregnant or breastfeeding.
18. Previous treatment with 230C9.
19. Previous exposure to fezakinumab (anti-IL-22 Ab).
20. Systemic treatment with immunosuppressive drugs (e.g. methotrexate, cyclosporine, azathioprine), immunomodulating drugs, retinoids (e.g. alitretinoin), corticosteroids (steroid eyedrops and inhaled or intranasal steroids are allowed), or JAK inhibitors within 28 days or 5 half-lives prior to baseline, whichever is longer.
21. Use of tanning beds or phototherapy (NBUVB, UVB, UVA1, PUVA), within 4 weeks prior to baseline.
22. Receipt of blood products within 28 days prior to screening.
23. Treatment with:
   Any marketed or investigational biologic agents within 3 months or 5 half-lives, whichever is longer, prior to baseline.
   Any cell-depleting agents including but not limited to rituximab: within 6 months prior to baseline, or until lymphocyte count returns to normal, whichever is longer.
24. Treatment with TCS, TCI, topical PDE-4 inhibitors, topical JAK inhibitors, or other medicated topical treatments within 7 days prior to baseline. NOTE: Subject may be rescreened (once) if failed for this criterion.
25. Receipt of live attenuated vaccines 30 days prior to baseline.
26. Treatment with any non-marketed drug substance (that is, an agent which has not yet been made available for clinical use following registration) within the last 4 weeks or 5 half-lives prior to randomization, whichever is longer.
27. Current participation in any other interventional clinical trial.
28. Employees of the trial site, or any other individuals directly involved with the planning or conduct of the trial, or immediate family members of such individuals.
29. Subjects who are legally institutionalized.

### Trial Design

The trial will consist of 3 periods (washout/screening, treatment, and follow-up).

For each subject the trial will last at least 33 weeks and up to 36 weeks, including:
A screening period of up to 4 weeks (from Week-4 up to Week 0) including an applicable washout period of 1 week for topical AD treatment (from Week-1 up to Week 0).
A treatment period of 16 weeks (from Week 0 up to Week 16) with subjects randomized into 1 of 4 active treatment groups or a placebo treatment group.
A follow-up period of 16 weeks (from Week 16 up to Week 32) for the assessment of efficacy, safety, PK, and ADAs.

Randomization will take place at Week 0 (Day 1, baseline). The primary endpoint will be assessed at Week 16. The final safety assessments will be conducted at Week 32 (end of trial).

### Screening Period (Between 0 and 4 Weeks Prior to the Baseline Visit [Day 1])

Eligibility of the subject to participate in the trial will be evaluated at a screening visit (Visit 1) and at the baseline visit (Visit 3). The screening visit will be performed up to 4 weeks before the baseline visit (Visit 3).

Moreover, at the screening visit (Visit 1), the subjects will receive training in completion of an eDiary, and will be given an electronic device to record the ADSD, Nocturnal Scratching, Difficulty Falling Asleep, and Frequency of Waking up During the Night. Completion of the eDiary twice daily will be initiated at the latest 1 week prior to the baseline visit (Visit 3, Week 0).

At the start of the washout period (from Visit 2, Week-1), subjects should stop any treatment with TCS, TCI, topical PDE-4 inhibitors, topical JAK inhibitors, or other medicated topical treatments.

Subjects should continue using their current daily skin care moisturizing routine from screening throughout the trial (until Week 32), if approved by the investigator, who will otherwise make an alternative suggestion.

### Randomization (Week 0)

At baseline (Visit 3, Week 0), eligibility of the subjects to participate in the trial must be confirmed. Eligible subjects will be randomized 1:1:1:1:1 to 1 of 5 treatment groups: 230 C9 (4 different dose regimens) or placebo. Randomization will be based on a permuted block design, stratified by baseline disease severity (baseline vIGA-AD score), region (Japan, Other countries), prior use of biologic for AD (Yes/No), and baseline biopsy status (willingness to provide biopsies: Yes/No). IRT will be used to control randomization and stratification factors.

Treatment period (from Week 0 up to Week 16)

In the treatment period, 230C9 and/or placebo injections will be administered at the site by unblinded site staff for 16 weeks, according to the treatment regimens.

During the treatment period, the subjects will be asked to continue applying their moisturizer, and to complete their eDiary twice daily. The subjects will also be asked to visit the clinic for assessments and procedures. Final dosing will be administered at Week 14. If medically necessary (i.e. due to intolerable AD signs and/or symptoms), rescue treatment for AD may be used from Week 4 at the discretion of the investigator (any rescue medication, regardless of type or class, used before Week 4 will lead to permanent discontinuation of IMP).

Follow-up period (from Week 16 up to Week 32)

The subject will have 4 follow-up visits at Weeks 20, 24, 28, and 32 for assessment of efficacy, safety, PK, and ADAs. Topical and systemic rescue treatment or procedures may be prescribed at the discretion of the investigator during the follow-up period, in order to control disease activity. In such case, the administered therapy will also be considered as rescue treatment, and subjects will continue the schedule of trial visits and assessments.

### Primary Endpoint

1. Percent change in EASI score from baseline to Week 16.

### Exploratory Endpoints (Efficacy)

Achievement of EASI-50 at Week 16.
Achievement of EASI-75 at Week 16.
Achievement of EASI-90 at Week 16.
Achievement of EASI-100 at Week 16.
Change in EASI score from baseline to Week 16.
Change in SCORAD score from baseline to Week 16.
Percent change in SCORAD score from baseline to Week 16.
Achievement of vIGA-AD score of 0 (clear) or 1 (almost clear) at Week 16.
Change in BSA from baseline to Week 16.
Percent change in BSA from baseline to Week 16.

### Exploratory Endpoints (PROs).

Reduction of ADSD Worst Itch score (weekly average)≥4 from baseline to Week 16.
Change in DLQI (and individual domain scores) from baseline to Week 16.
Percent change in DLQI (and individual domain scores) from baseline to Week 16.
Change in POEM score from baseline to Week 16.
Percentage change in POEM score from baseline to Week 16.
Change in EQ-5D-5L index score from baseline to Week 16.
Percent change in EQ-5D-5L index score from baseline to Week 16.
Change in EQ-5D-5L VAS score from baseline to Week 16.
Percent change in EQ-5D-5L VAS score from baseline to Week 16.
Change in HADS score from baseline to Week 16.
Percent change in HADS score from baseline to Week 16.
Change in Difficulty Falling Asleep (weekly average) from baseline to Week 16.
Percent change in Difficulty Falling Asleep (weekly average) from baseline to Week 16.
Change in Nocturnal Scratching (weekly average) from baseline to Week 16.
Percent change in Nocturnal Scratching (weekly average) from baseline to Week 16.
Change in Frequency of Waking up During the Night (weekly average) from baseline to Week 16.
Percent change in Frequency of Waking up During the Night (weekly average) from baseline to Week 16.
Change in WPAI-SHP domain scores from baseline to Week 16.
Percent change in WPAI-SHP domain scores from baseline to Week 16.
Change in SCORAD Part C (Pruritus VAS) score from baseline to Week 16.
Percent change in SCORAD Part C (Pruritus VAS) score from baseline to Week 16.
Reduction of ADSD Worst Skin Pain score (weekly average)≥4 from baseline to Week 16.
Reduction of DLQI≥4 from baseline to Week 16.
Reduction of POEM≥4 from baseline to Week 16.
Time from baseline to use of any rescue medication.

1. An IL-22R binding protein for use in a method of treating atopic dermatitis (AD) in a subject, wherein the method comprises the steps of: (a) administering subcutaneously one or more loading dose(s) of the IL-22R binding protein to the subject; and (b) administering subcutaneously one or more secondary dose(s) of the IL-22R binding protein to the subject, wherein the II22R binding protein is an antibody comprising a HCDR1 having the amino acid sequence of SEQ ID no. 1 (SYDMN), a HCDR2 having the amino acid sequence of SEQ ID no.2 (SIYNDASNTAYSDSVKG), a HCDR3 having the amino acid sequence of SEQ ID no. 3 (VGFSGTYYSES), a LCDR1 having the amino acid sequence of SEQ ID no 4 (QGGYYAH), a LCDR2 having the amino acid sequence of SEQ ID No 5 (GQNNRPS) and a LCDR3 having the amino acid sequence of SEQ ID no 6 (QSGSSSSNAV).

2. A method of treating atopic dermatitis (AD) in a subject in need thereof, wherein the method comprises the steps of: (a) administering subcutaneously one or more loading dose(s) of an IL-22R binding protein to the subject; and (b) administering subcutaneously one or more secondary dose(s) of the IL-22R binding protein to the subject, wherein the 1122R binding protein is an antibody comprises a HCDR1 having the amino acid sequence of SEQ ID no.1 (SYDMN), a HCDR2 having the amino acid sequence of SEQ ID no.2 (SIYNDASNTAYSDSVKG), a HCDR3 having the amino acid sequence of SEQ ID no.3 (VGFSGTYYSES), a LCDR1 having the amino acid sequence of SEQ ID no 4 (QGGYYAH), a LCDR2 having the amino acid sequence of SEQ ID No 5 (GQNNRPS) and a LCDR3 having the amino acid sequence of SEQ ID no 6 (QSGSSSSNAV).

3. Use of an IL-22R binding protein in the manufacture of a medicament for treating atopic dermatitis (AD) in a subject, wherein the method for treating AD comprises the steps of: (a) administering subcutaneously one or more loading dose(s) of the IL-22R binding protein to the subject; and (b) administering subcutaneously one or more secondary dose(s) of the IL-22R binding protein to the subject, wherein the II22R binding protein is an antibody comprising a HCDR1 having the amino acid sequence of SEQ ID no. 1 (SYDMN), a HCDR2 having the amino acid sequence of SEQ ID no.2 (SIYNDASNTAYSDSVKG), a HCDR3 having the amino acid sequence of SEQ ID no.3 (VGFSGTYYSES), a LCDR1 having the amino acid sequence of SEQ ID no 4 (QGGYYAH), a LCDR2 having the amino acid sequence of SEQ ID No 5 (GQNNRPS) and a LCDR3 having the amino acid sequence of SEQ ID no 6 (QSGSSSSNAV).

4. The IL-22R binding protein for use, the method or the use according to any one of the preceding claims, wherein the 1I22R binding is an antibody comprises a VH having the amino acid sequence of SEQ ID NO 7 (QVQLVESGGG LVQPGGSLRL SCAASGFTFS SYDMNWVRQA PGKGLEWVSS IYNDASNTAY SDSVKGRFTI SRDNSKNTLY LQMNSLRAED TAVYYCAKVG FSGTYYSESW GQGTLVTVSS) and a VL having the amino acid sequence of SEQ ID NO. 8 (SYELTQPSSV SVALGQTARI TCQGGYYAHW YQQKPGQAPV LVIYGQNNRP SGIPERFSGS GAGNTATLTI SRAQAEDEAD YYCQSGSSSS NAVFGGGTKLTVL).

5. The IL-22R binding protein for use, the method or the use according to any one of the preceding claims, wherein the 1I22R binding is an antibody comprises a HC having the amino acid sequence of SEQ ID NO 9 (QVQLVESGGG LVQPGGSLRL SCAASGFTFS SYDMNWVRQA PGKGLEWVSS IYNDASNTAY SDSVKGRFTI SRDNSKNTLY LQMNSLRAED TAVYYCAKVG FSGTYYSESW GQGTLVTVSS ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKVEP KSCDKTHTCP PCPAPELLGG PSVFLFPPKP KDTLMISRTP EVTCVVVDVS HEDPEVKFNW YVDGVEVHNA KTKPREEQYQ STYRVVSVLT VLHQDWLNGK EYKCKVSNKA LPAPIEKTIS KAKGQPREPQ VYTLPPSRDE LTKNQVSLTC LVKGFYPSDI AVEWESNGQP ENNYKTTPPV LDSDGSFFLY SKLTVDKSRW QQGNVFSCSV MHEALHNHYT QKSLSLSPGK) and a LC having the amino acid sequence of SEQ ID NO. 10 (SYELTQPSSV SVALGQTARI TCQGGYYAHW YQQKPGQAPV LVIYGQNNRP SGIPERFSGS GAGNTATLTI SRAQAEDEAD YYCQSGSSSS NAVFGGGTKL TVLGQPKAAP SVTLFPPSSE ELQANKATLV CLISDFYPGA VTVAWKADSS PVKAGVETTT PSKQSNNKYA ASSYLSLTPE QWKSHRSYSC QVTHEGSTVE KTVAPTECS).

6. The IL-22R binding protein for use, the method or the use according to any one of the preceding claims, wherein the method comprises the steps of:
(a) administering one or more loading dose(s) of about 600 mg of the IL-22R binding protein to the subject; and (b) administering one or more secondary dose(s) of about 600 mg of the IL-22R binding protein to the subject, wherein each secondary dose is administered to the subject about 2 weeks after the immediately preceding dose;
(b) administering one or more loading dose(s) of about 450 mg of the IL-22R binding protein to the subject; and (b) administering one or more secondary dose(s) of about 450 mg of the IL-22R binding protein to the subject, wherein each secondary dose is administered to the subject about 2 weeks after the immediately preceding dose;
(c) administering one or more loading dose(s) of about 600 mg of the IL-22R binding protein to the subject; and (b) administering one or more secondary dose(s) of about 300 mg of the IL-22R binding protein to the subject, wherein each secondary dose is administered to the subject about 2 weeks after the immediately preceding dose; or
(d) administering one or more loading dose(s) of about 300 mg of the IL-22R binding protein to the subject; and (b) administering one or more secondary dose(s) of about 150 mg of the IL-22R binding protein to the subject, wherein each secondary dose is administered to the subject about 2 weeks after the immediately preceding dose.

7. The IL-22R binding protein for use, the method or the use according to any one of the preceding claims, wherein the method comprises the steps of:
(a) administering five loading dose(s) of about 600 mg of the IL-22R binding protein to the subject; and (b) administering one or more secondary dose(s) of about 600 mg of the IL-22R binding protein to the subject, wherein each loading dose after the first loading dose is administered to the subject about 1 week after the immediately preceding loading dose and each secondary dose is administered to the subject about 2 weeks after the immediately preceding dose;
(b) administering three loading dose(s) of about 450 mg of the IL-22R binding protein to the subject; and (b) administering one or more secondary dose(s) of about 450 mg of the IL-22R binding protein to the subject, wherein each loading dose is administered to the subject about 1 week after the immediately preceding loading dose and wherein each secondary dose is administered to the subject about 2 weeks after the immediately preceding dose;
(c) administering two loading doses of about 600 mg of the IL-22R binding protein to the subject; and (b) administering one or more secondary dose(s) of about 300 mg of the IL-22R binding protein to the subject, wherein the second loading dose is administered to the subject about 2 weeks after the first loading dose and wherein each secondary dose is administered to the subject about 2 weeks after the immediately preceding dose; or
(d) administering two loading doses of about 300 mg of the IL-22R binding protein to the subject; and (b) administering one or more secondary dose(s) of about 150 mg of the IL-22R binding protein to the subject, wherein the second loading dose is administered to the subject about 2 weeks after the first loading dose and wherein each secondary dose is administered to the subject about 2 weeks after the immediately preceding dose.

8. The IL-22R binding protein for use, the method or the use according to any one of the preceding claims, wherein step (b) is continued for at least 8 weeks, at least 12 weeks, at least 3 months, at least 16 weeks, at least 20 weeks, at least 24 weeks, at least 6 months, at least 28 weeks, at least 32 weeks, at least 36 weeks, at least 44 weeks, at least a year, or at least 52 weeks or more.

9. The IL-22R binding protein for use, the method or the use according to any one of the preceding claims, wherein the subject's has moderate to severe atopic dermatitis as defined by the Hanifin and Rajka (1980) criteria for AD and a history for AD≥1 year.

10. The IL-22R binding protein for use, the method or the use according to any one of the preceding claims, wherein the patient has
EASI score≥16 at baseline,
vIGA-AD score≥3 at baseline,
BSA of AD involvement≥10 at baseline,
and/or
ADSD worst itch score (weekly average)≥4 at baseline.

11. The IL-22R binding protein for use, the method or the use according to any one of the preceding claims, wherein the subject has a recent history (within 12 months before screening) with documented inadequate response to treatment with TCS (±TCI as appropriate) or for whom these topical AD treatments are medically inadvisable e.g. due to important side effects or safety risks

12. The IL-22R binding protein for use, the method or the use according to any one of the preceding claims, wherein administration of the IL-22R antibody eliminates or reduces the subjects disease severity and wherein the disease severity is assessed by an Atopic Dermatitis Disease Severity Outcome Measure.

13. The IL-22R binding protein for use, the method or the use according to claim 12, wherein Atopic Dermatitis Disease Severity Outcome Measure is Eczema Area and Severity Index (EASI) and the subjects EASI score is reduced from baseline.

14. The IL-22R binding protein for use, the method or the use according to claim 12, wherein Atopic Dermatitis Disease Severity Outcome Measure is Eczema Area and Severity Index (EASI) and the subjects EASI score is reduced from baseline following 16 weeks of treatment or earlier.

15. The IL-22R binding protein for use, the method or the use according to claim 12, wherein Atopic Dermatitis Disease Severity Outcome Measure is the Validated Investigator Global Assessment Scale for Atopic Dermatitis (vIGA-AD) and the subject achieves a vIGA-AD score of 0 (clear) or 1 (almost clear) at week 16 or earlier.

16. The IL-22R binding protein for use, the method or the use according to claim 12, wherein Atopic Dermatitis Disease Severity Outcome Measure is Severity Scoring of Atopic Dermatitis (SCORAD9 and the subjects SCORAD score is reduced from baseline.

17. The IL-22R binding protein for use, the method or the use according to claim 12, wherein Atopic Dermatitis Disease Severity Outcome Measure is affected Body Surface Area (BSA) and the subjects BSA score is reduced from baseline.

18. The IL-22R binding protein for use, the method or the use according to claim 12, wherein Atopic Dermatitis Disease Severity Outcome Measure is a Patient reported outcome POEM, POEM, EQ-5D-5L index, EQ-5D-5L VAS, WPAI-SHP, HADS, SCORAD Part C (pruritus VAS). ADSD Worst Itch and ADSD worst skin pain and the patient reported outcome is reduced from baseline.

19. The IL-22R binding protein for use, the method or the use according to any of the preceding claims, wherein each dose of the IL-22R binding protein is administered as a pharmaceutical composition comprising comprising 150±15 mg/mL-225 mg/ml±25 mg/ml of IL-22R binding protein, one or more disaccharides, one or more amino acids, optionally a surfactant and at a pH of 5.5-6.5 and with an osmolality of 280-450 mOsm/kg.

20. The IL-22R binding protein for use, the method or the use according to claim 20, wherein each dose of the IL-22R binding protein is administered as a pharmaceutical composition comprising comprising about 150 mg/ml of the IL-22R binding protein, about 20 mM Histidine, about 80 mM Glycine, about 20 mM Methionine, about 180 mM Trehalose, optionally 20 mg/ml Tween 20 at a pH of 6.3 and with an Osmolality of 331 (mOsm/Kg).

21. The IL-22R binding protein for use, the method or the use according to any one of the preceding claims, wherein the IL-22R binding protein is administered as a monotherapy.

22. The IL-22R binding protein for use, the method or the use according to any one of the preceding claims, wherein the IL-22R binding protein is administered in combination with a second therapeutic agent selected from the group consisting of a topical corticosteroid, a topical calcineurin inhibitor, an anti-histamine, an emollient, or an anti-bacterial therapeutic.


SEQUENCE LISTING (10 sequences)

SEQ ID NO: 1 - protein, 5 aa, synthetic construct
SYDMN

SEQ ID NO: 2 - protein, 17 aa, synthetic construct
SIYNDASNTAYSDSVKG

SEQ ID NO: 3 - protein, 11 aa, synthetic construct
VGFSGTYYSES

SEQ ID NO: 4 - protein, 7 aa, synthetic construct
QGGYYAH

SEQ ID NO: 5 - protein, 7 aa, synthetic construct
GQNNRPS

SEQ ID NO: 6 - protein, 10 aa, synthetic construct
QSGSSSSNAV

SEQ ID NO: 7 - protein, 120 aa, synthetic construct
QVQLVESGGGLVQPGGSLRLSCAASGFTFSSYDMNWVRQAPGKGLEWVSSIYNDASNTAYSDSVKGRFTISRDNSKNTLYLQMNSLRAEDTAVYYCAKVGFSGTYYSESWGQGTLVTVSS

SEQ ID NO: 8 - protein, 103 aa, synthetic construct
SYELTQPSSVSVALGQTARITCQGGYYAHWYQQKPGQAPVLVIYGQNNRPSGIPERFSGSGAGNTATLTISRAQAEDEADYYCQSGSSSSNAVFGGGTKLTVL

SEQ ID NO: 9 - protein, 450 aa, synthetic construct
QVQLVESGGGLVQPGGSLRLSCAASGFTFSSYDMNWVRQAPGKGLEWVSSIYNDASNTAYSDSVKGRFTISRDNSKNTLYLQMNSLRAEDTAVYYCAKVGFSGTYYSESWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 209 aa, synthetic construct
SYELTQPSSVSVALGQTARITCQGGYYAHWYQQKPGQAPVLVIYGQNNRPSGIPERFSGSGAGNTATLTISRAQAEDEADYYCQSGSSSSNAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS